SEQUENCE LISTING (8 sequences)

SEQ ID NO: 1 - protein, 252 aa, Artificial Sequence
  Peptide sequence MecA3136
MHHHHHHSSGLVPCGWLKETAAAKFERQHMDSPDLSTWSHPQFEKGGGSGGGSGGGSWSHPQFEKGTGSGGGSGGGSWSHPQFEKGTGSGGGSGGGSWSHPQFEKGTGSGGGSGGGSWSHPQFEKGTDDDDKAMADIGSEMIPRGLSEAKPATPEIQEIVDKVKPQLEEKTNETYGKLEAVQYKTQVLAKILTLGSTNYYIKVRAGDNKYMHLKVFKSLALELSETPTPKAADRVLTGYQVDKNKDDELTGF

SEQ ID NO: 2 - protein, 246 aa, Artificial Sequence
  Peptide sequence MecA3140
MHHHHHHSSGLVPCGWLKETAAAKFERQHMDSPDLSTWSHPQFEKGGGSGGGSGGGSWSHPQFEKGTGSGGGSGGGSWSHPQFEKGTGSGGGSGGGSWSHPQFEKGTGSGGGSGGGSWSHPQFEKGTDDDDKAMADIGSEMIPRGLSEAKPATPEIQEIVDKVKPQLEEKTNETYGKLEAVQYKTQVLASTNYYIKVRAGDNKYMHLKVFKSLFVVIQPERSNTWADRVLTGYQVDKNKDDELTGF

SEQ ID NO: 3 - protein, 246 aa, Artificial Sequence
  Peptide sequence HIW
MHHHHHHSSGLVPCGWLKETAAAKFERQHMDSPDLSTWSHPQFEKGGGSGGGSGGGSWSHPQFEKGTGSGGGSGGGSWSHPQFEKGTGSGGGSGGGSWSHPQFEKGTGSGGGSGGGSWSHPQFEKGTDDDDKAMADIGSEMIPRGLSEAKPATPEIQEIVDKVKPQLEEKTNETYGKLEAVQYKTQVLASTNYYIKVRAGDNKYMHLKVFKSLHIWPITEIRRLVADRVLTGYQVDKNKDDELTGF

SEQ ID NO: 4 - protein, 115 aa, Artificial Sequence
  Peptide sequence E1
ASAATGVRAVPGNENSLEIEELARFAVDEHNKKENALLEFVRVVKAKEQHWKAKLGHDTMYYLTLEAKDGGKKKLYEAKVWVKLIPTKDFHLNFKELQEFKPVGDAAAAHHHHHH

SEQ ID NO: 5 - protein, 252 aa, Artificial Sequence
  Peptide sequence Red1
MHHHHHHSSGLVPCGWLKETAAAKFERQHMDSPDLSTWSHPQFEKGGGSGGGSGGGSWSHPQFEKGTGSGGGSGGGSWSHPQFEKGTGSGGGSGGGSWSHPQFEKGTGSGGGSGGGSWSHPQFEKGTDDDDKAMADIGSEMIPRGLSEAKPATPEIQEIVDKVKPQLEEKTNETYGKLEAVQYKTQVLANLVGRISTNYYIKVRAGDNKYMHLKVFNGPRQIKQVEWELIWADRVLTGYQVDKNKDDELTGF

SEQ ID NO: 6 - protein, 224 aa, Artificial Sequence
  Peptide sequence Red2
MHHHHHHSSGLVPCGWLKETAAAKFERQHMDSPDLSTWSHPQFEKGGGSGGGSGGGSWSHPQFEKGTGSGGGSGGGSWSHPQFEKGTGSGGGSGGGSWSHPQFEKGTGSGGGSGGGSWSHPQFEKGTDDDDKAMADIGSEMIPRGLSEAKPATPEIQEIVDKVKPQLEEKTNETYGKLEAVQYKTQVLANLVGRISTNYYIKVRAGDNKYMHLKVFKSLTPLEL

SEQ ID NO: 7 - DNA, 54 bp, Artificial Sequence
  Forward DNA primer
ggatccgaattcctggaagttctgttccaggggcccatggcttcaaaagataaa

SEQ ID NO: 8 - DNA, 30 bp, Artificial Sequence
  Reverse DNA primer
aagcttctcgagttattcatctatatcgta

(19) United States
(12) Patent Application Publication
(10) Pub. No.: US 2016/0089393 A1
(43) Pub. Date: Mar. 31, 2016

(54) COMPOSITION AND METHODS FOR COMBATING ANTIBACTERIAL RESISTANT BACTERIA

(71) Applicant: BLUEBERRY THERAPEUTICS LIMITED, Macclesfield, Cheshire (GB)

(72) Inventors: John Ridden, Leek (GB); Liam Good, London (GB)

(21) Appl. No.: 14/785,076

(22) PCT Filed: Apr. 17, 2014
(86) PCT No.: PCT/GB2014/051206

§ 371 Date: Oct. 16, 2015

(30) Foreign Application Priority Data
Apr. 17, 2013 (GB) 1306980.2

(51) Int. Cl. A61K 31/785; A61K 45/06

(52) U.S. Cl. CPC A61K 31/785; A61K 45/06

(57) ABSTRACT

The present invention provides compositions and methods for sensitizing a bacterium to an antibacterial the method comprising the step of exposing the bacterium, in the presence of an entry-promoting agent, to an agent that inhibits antibacterial resistance. Methods for killing an antibacterial-resistant bacterium comprising the step of exposing the bacterium, in the presence of an entry-promoting agent, to an agent that inhibits antibacterial resistance and exposing the bacterium to an antibacterial are also provided. The methods may also include the step of identifying the bacterium and the antibacterial resistance profile of the bacterium and tailoring the methods accordingly. Exemplary entry promoting agents provided are polyhexamethylene biguanide (PHMB) and polyhexamethyleneguanide (PHMG). Exemplary agents that inhibit antibacterial resistance are agents that bindto and inhibit antibacterial-resistance determinants such as beta-lactamases, PBP2a, NDM-1 and Vim2. Compositions, pharmaceutical compositions/formulations and medical uses thereof are also provided according to the invention.

The present invention relates to a composition and methods for combating antibacterial resistant bacteria. In particular it relates to methods for sensitizing a bacterium to an antibacterial and exposing the bacterium to the antibacterial to which it has been sensitized in order to kill it.

Bacteria cause some of the most severe human and animal infectious diseases and remain a major cause of mortality throughout the world. The development of antibacterial resistance is a major problem in the treatment of bacterial infections in the hospital and in the community. There are different mechanisms of resistance to antimicrobials, such as intrinsic resistance which is a consequence of general adaptive processes that are not necessarily linked to a given class of antimicrobial. An example of such resistance may be found in Pseudomonas aeruginosa, whose low membrane permeability is likely to be the main reason for its innate resistance to many antibacterials. Acquired resistance is the major mechanism of active antimicrobial resistance and is the result of a specific evolutionary pressure to allow bacteria previously sensitive to antimicrobials to become resistant. Such resistance includes changes to permeability barriers including overexpression of efflux pumps, alterations to the target of the antimicrobial drug, and inactivation of the antibacterial, for example by enzymatic modification. Antimicrobial resistance in bacteria is reviewed in Bockstael & Van Aerschot (2009) Eur. J. Med. 4(2), 141-155.

There remains the need for further methods of combating bacterial infection, and the present invention is directed to methods that sensitize antibacterial resistant bacteria to the effect of an antibacterial, thereby helping to overcome antibacterial resistance. Agents that inhibit antibacterial resistance determinants are known but not all have proved useful in sensitizing bacteria to antibacterials because of their inability to enter the antibacterial resistant bacterium and reach the target site. The invention aims to overcome some of these difficulties.

A first aspect of the invention provides for a composition comprising a bacterium entry-promoting agent and an agent that inhibits antibacterial resistance.

The composition may further comprise an antibacterial.

A further aspect of the invention provides a method for sensitizing a bacterium to an antibacterial the method comprising the step of exposing the bacterium, in the presence of an entry-promoting agent, to an agent that inhibits antibacterial resistance.

An additional aspect of the invention provides a method for killing an antibacterial-resistant bacterium the method comprising the step of exposing the bacterium, in the presence of an entry-promoting agent, to an agent that inhibits antibacterial resistance and exposing the bacterium to an antibacterial.

The entry-promoting agent in any aspect or embodiment of the present invention is preferably an entry-promoting agent as described in PCT/GB2012/052526, filed 11 Oct. 2012, incorporated herein by reference.

The term “entry-promoting agent” is intended to mean a compound or compositions which enables entry to the capsule and/or cell wall of a bacterium. Preferably, the agent further enables an agent that inhibits antibacterial resistance to also pass through the cytoplasmic membrane of a bacterium.

### Entry-Promoting Agent

The entry-promoting agent of any aspect or embodiment of the present invention is typically a polymer, wherein the polymer comprises a linear and/or branched polymonoguanide/polyguanidine, polybiguanide, analogue or derivative thereof, for example according to the following Formula 1a or Formula 1b, with examples given in tables 1 and 2, below:

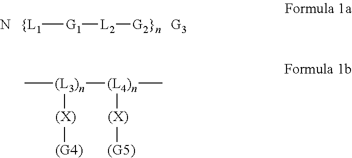

wherein:
“n”, refers to number of repeating units in the polymer, and n can vary from 2 to 1000, for example from 2 or 5 to 10, 20, 30, 40, 50, 60, 70, 80, 90, 100, 150, 200, 250, 300, 350, 400, 450, 500, 600, 700, 800 or 900;
G₁ and G₂ independently represent a cationic group comprising biguanide or guanidine, wherein L₁ and L₂ are directly joined to a Nitrogen atom of the guanide. Thus, the biguanide or guanidine groups are integral to the polymer backbone. The biguanide or guanidine groups are not side chain moieties in formula 1a.

Example of Cationic Groups:

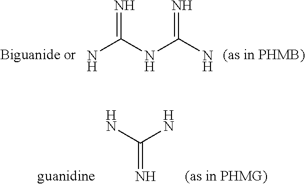

In the present invention, L₁ and L₂ are the linking groups between the G₁ and G₂ cationic groups in the polymer. L₁ and L₂ can independently represent an aliphatic group containing C₁-C₁₄₀ carbon atoms, for example an alkyl group such as methylene, ethylene, propylene, C₄, C₅, C₆, C₇, C₈, C₉ or C₁₀; C₁-C₁₀, -C₂₀, -C₃₀, -C₄₀, -C₅₀ -C₆₀, -C₇₀, -C₈₀, -C₉₀, -C₁₀₀, -C₁₁₀, -C₁₂₀, -C₁₃₀ or -C₁₄₀, alkyl; or L₁ and L₂ can (independently) be C₁-C₁₄₀ (for example C₁, C₂, C₃, C₄, C₅, C₆, C₇, C₈, C₉ or C₁₀; C₁-C₁₀, -C₂₀, -C₃₀, -C₄₀, -C₅₀ -C₆₀, -C₇₀, -C₈₀, -C₉₀, -C₁₀₀, -C₁₁₀, -C₁₂₀, -C₁₃₀ or -C₁₄₀), cycloaliphatic, heterocyclic, aromatic, aryl, alkylaryl, arylalkyl, oxyalkylene radicals, or L₁ and L₂ can (independently) be a polyalkylene radical optionally interrupted by one or more, preferably one, oxygen, nitrogen or sulphur atoms, functional groups as well as saturated or unsaturated cyclic moiety. Examples of suitable L₁ and L₂ are groups are listed in table 1.

L₁, L₂, G₁ and G₂ may have been modified using aliphatic, cycloaliphatic, heterocyclic, aryl, alkaryl, and oxyalkylene radicals.

N and G₃ are preferably end groups. Typically the polymers of use in the invention have terminal amino (N) and cyanoguanidine (G₃) or guanidine (G₃) end groups. Such end groups may be modified (for example with 1,6-diaminohexane, 1,6 di(cyanoguanidino)hexane, 1,6-diguanidinohexane, 4-guanidinobutyric acid) by linkage to aliphatic, cycloaliphatic heterocyclic heterocyclic, aryl, alkylaryl, arylalkyl, oxyalkylene radicals. In addition, end groups may be modified by linkage to receptor ligands, dextrans, cyclodextrins, fatty acids or fatty acid derivatives, cholesterol or cholesterol derivatives or polyethylene glycol (PEG). Optionally, the polymer can end with guanidine or biguanide or cyanoamine or amine or cyanoguanidine at N and G₃ positions or cyanoamine at N and cyanoguanidine at G₃ position or guanidine at N and Cyanoguanidne at G₃ positions or L1 amine at G₃ and cyanoguanidine at N. G₃ can be L₁-amine, L₂-cyanoguanidine or L₂-guanidine. Depending on the number of polymerization (n) or polymer chain breakage and side reactions during synthesis, heterogeneous mixture of end groups can arise as described above as an example. Thus, the N and G3 groups can be interchanged/present as a heterogeneous mixture, as noted above. Alternatively N and G₃ may be absent and the polymer may be cyclic, in which case the respective terminal L₁ and G₂ groups are linked directly to one another.

In formula 1 b, X can be either present or absent. L₃, L₄ and X are as noted above for “L₁ or L₂”. In Thus, L₃ and L₄ and X are the linking groups between the G₄ and G₅ cationic groups in the polymer. L₃ and L₄ and X can independently represent an aliphatic group containing C₁-C₁₄₀ carbon atoms, for example an alkyl group such as methylene, ethylene, propylene, C₄, C₅, C₆, C₇, C₈, C₉ or C₁₀; C₁-C₁₀, -C₂₀, -C₃₀, -C₄₀, -C₅₀ -C₆₀, -C₇₀, -C₈₀, -C₉₀, -C₁₀₀, -C₁₁₀, -C₁₂₀, -C₁₃₀ or -C₁₄₀, alkyl; or L₃ and L₄ and X can independently be C₁-C₁₄₀ (for example C₁, C₂, C₃, C₄, C₅, C₆, C₇, C₈, C₉ or C₁₀; C₁-C₁₀, -C₂₀, -C₃₀, -C₄₀, -C₅₀ -C₆₀, -C₇₀, -C₈₀, -C₉₀, -C₁₀₀, - C₁₁₀, -C₁₂₀, -C₁₃₀ or -C₁₄₀), cycloaliphatic, heterocyclic, aromatic, aryl, alkylaryl, arylalkyl, oxyalkylene radicals, or L₃ and L₄ and X can independently be a polyalkylene radical optionally interrupted by one or more, preferably one, oxygen, nitrogen or sulphur atoms, functional groups as well as saturated or unsaturated cyclic moiety. Examples of suitable L₃ and L₄ and X are groups are listed in table 2.

“G₄” and “G₅” are cationic moieties and can be same or different. At least one of them is a biguanidine moiety or carbamoylguanidine, and the other moiety may be as above (biguanidine or carbamoylguanidine) or amine. For the avoidance of doubt, in formula 1 b, cationic moiety G₄ and G₅ do not contain only single guanidine groups. For example, G₄ and G₅ typically do not contain single guanidine groups. Examples of such compounds are polyallylbiguanide, poly(allylbiguanidnio-co-allylamine), poly(allylcarbamoylguanidino-co-allylamine), polyvinylbiguanide, as listed in table 2.

Example of polyallylbiguanide is as shown below

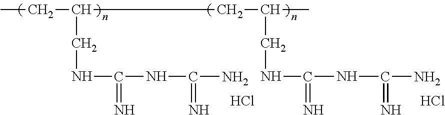 [SMILES: CCC(CNC(=N)NC(=N)N)CC(C)CNC(=N)NC(=N)N ; Cl ; Cl]

In case of polyallylbigunidine L₃ and L₄ are identical, G₄ and G₅ are similar, thus polyallylbiguanide can be simplified as below.

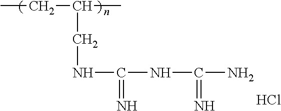

Example of poly(allylcarbamoylguanidnio-co-allylamine) is as shown below

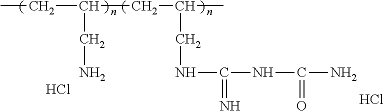

The polymers for use in the invention will generally have counter ions associated with them. Suitable counter ions include but are not limited to the following: halide (for example chloride), phosphate, lactate, phosphonate, sulfonate, amino carboxylate, carboxylate, hydroxy carboxylate, organophosphate, organophosphonate, organosulfornate and organosuflate.

Polymers for use in the invention can be either heterogeneous mixtures of polymers of different “n” number or homogenous fractions comprising specified “n” numbers purified by standard purification methods. As indicated above the polymers may also be cyclic and in addition may be branched.

Preferred numbers for “n” include 2-250, 2-100, 2-80 and 2-50.

TABLE 1
Examples of polymer analogues arising from Formula 1a.

| Name | L₁ | G₁ | L₂ | G₂ |
|---|---|---|---|---|
| Polyhexamethylene biguanide | (CH₂)₆ | Biguanide | (CH₂)₆ | Biguanide |
| (PHMB) | | | | |
| Polyethylene biguanide (PEB) | (CH₂)₂ | Biguanide | (CH₂)₂ | Biguanide |
| Polyethylenetetramethylene | (CH₂)₂ | Biguanide | (CH₂)₄ | Biguanide |
| biguanide | | | | |
| Polyethylene hexamethylene | (CH₂)₂ | Biguanide | (CH₂)₆ | Biguanide |
| biguanide (PEHMB) | | | | |
| Polypropylene biguanide, | (CH₂)₃ | Biguanide | (CH₂)₃ | Biguanide |
| Polyaminopropyl biguanide (PAPB) | | | | |
| Poly-[2-(2-ethoxy)-ethoxyethyl]- | (CH₂CH₂OCH₂CH₂OCH₂CH₂) | Biguanide | (CH₂CH₂OCH₂CH₂OCH₂CH₂) | Biguanide |
| biguanide-chloride] | | | | |
| (PEEG) | | | | |
| Polypropylenehexamethylene | (CH₂)₃ | Biguanide | (CH₂)₆ | Biguanide |
| biguanide | | | | |
| Polyethyleneoctamethylene | (CH₂)₂ | Biguanide | (CH₂)₈ | Biguanide |
| biguanide | | | | |
| Polyethylenedecamethylene | (CH₂)₂ | Biguanide | (CH₂)₁₀ | Biguanide |
| biguanide | | | | |
| Polyethylenedodecamethylene | (CH₂)₂ | Biguanide | (CH₂)₁₂ | Biguanide |
| biguanide | | | | |
| Polytetramethylenehexamethylene | (CH₂)₄ | Biguanide | (CH₂)₆ | Biguanide |
| biguanide | | | | |
| Polytetramethylenebiguanide | (CH₂)₄ | Biguanide | (CH₂)₄ | Biguanide |
| Polypropyleneoctamethylene | (CH₂)₃ | Biguanide | (CH₂)₈ | Biguanide |
| biguanide | | | | |
| Polytetramethyleneoctamethylene | (CH₂)₄ | Biguanide | (CH₂)₈ | Biguanide |
| Biguanide | | | | |
| Polyhexamethylene | (CH₂)₆ | Biguanide | CH₂—CH₂—NH—CH₂—CH₂ | Biguanide |
| diethylenetriamine biguanide | | | | |
| Polyhexamethylene guanide | (CH₂)₆ | guanidine | (CH₂)₆ | guanidine |
| (PHMG) | | | | |
| Polyethylene guanide | (CH₂)₂ | guanidine | (CH₂)₂ | guanidine |
| Polyethylenetetramethylene guanide | (CH₂)₂ | guanidine | (CH₂)₄ | guanidine |
| Polyethylene hexamethylene | (CH₂)₂ | guanidine | (CH₂)₆ | guanidine |
| guanide | | | | |
| Polypropylene guanide, | (CH₂)₃ | guanidine | (CH₂)₃ | guanidine |
| Polyaminopropyl guanide (PAPB) | | | | |
| Poly-[2-(2-ethoxy)-ethoxyethyl]- | (CH₂CH₂OCH₂CH₂OCH₂CH₂) | guanidine | (CH₂CH₂OCH₂CH₂OCH₂CH₂) | guanidine |
| guanide | | | | |
| Polypropylenehexamethylene | (CH₂)₃ | guanidine | (CH₂)₆ | guanidine |
| guanide | | | | |
| Polyethyleneoctamethylene | (CH₂)₂ | guanidine | (CH₂)₈ | guanidine |
| guanide | | | | |
| Polyethylenedecamethylene | (CH₂)₂ | guanidine | (CH₂)₁₀ | guanidine |
| guanide | | | | |
| Polyethylenedodecamethylene | (CH₂)₂ | guanidine | (CH₂)₁₂ | guanidine |
| guanide | | | | |
| Polytetramethylenehexamethylene | (CH₂)₄ | guanidine | (CH₂)₆ | guanidine |
| guanide | | | | |
| Polypropyleneoctamethylene | (CH₂)₃ | guanidine | (CH₂)₈ | guanidine |
| guanide | | | | |
| Polytetramethylene guanide | (CH₂)₄ | guanidine | (CH₂)₄ | guanidine |
| Polyhexamethylene | (CH₂)₆ | guanidine | CH₂—CH₂—NH—CH₂—CH₂ | guanidine |
| diethylenetriamine guanide | | | | |

CAS Numbers for Example Compounds Arising from Formula 1a

| Polymer | CAS Number |
|---|---|
| Polyhexamethylene biguanide hydrochloride (PHMB) | 27083-27-8 |
| | 32289-58-0 |
| Polyhexamethylene guanidine hydrochloride (PHMG) | 57028-96-3 |
| Poly-[2-(2-ethoxy)-ethoxyethyl]-guanidinium-chloride] | 374572-91-5 |
| (PEEG) | |

TABLE 2
Examples of polymer analogues arising from Formula 1b.

| Name | L₃ | G₄ | L₄ | G₅ | X |
|---|---|---|---|---|---|
| Polyallylbiguanide | (CH₂—CH) | Biguanide | (CH₂—CH) | Biguanide | CH₂ |
| poly(allylbiguanidnio-co- | (CH₂—CH) | amine | (CH₂—CH) | biguanide | CH₂ |
| allylamine) | | | | | |
| poly(allylcarbamoylguanidino- | (CH₂—CH) | amine | (CH₂—CH) | Carbamoyl | CH₂ |
| co-allylamine) | | | | guanidine | |
| polyvinylbiguanide | (CH₂—CH) | Biguanide | (CH₂—CH) | biguanide | absent |

The entry-promoting agent used in the methods, compositions, formulations, uses and kits of the invention may comprise linear, branched or dendrimeric molecules. The entry promoting agent may comprise a combination of linear, branched or dendrimeric molecules. The entry promoting agent may comprise one or any combination of molecules of Formula 1a or formula 1b, for example as described above.

For example, the entry-promoting agent can comprise one or more of polyhexamethylene biguanide (PHMB), polyhexamethylene monoguanide (PHMG), polyethylene biguanide (PEB), polytetramethylene biguanide (PTMB) or polyethylene hexamethylene biguanide (PEHMB). Some examples are listed in tables 1 and 2. The entry-promoting agent of any aspect of the invention is preferably PHMB or PHMG or an analogue or derivative either thereof.

Thus, the entry-promoting agent may comprise homogeneous or heterogeneous mixtures of one or more of polyhexamethylene biguanide (PHMB), polyhexamethylene monoguanide (PHMG), polyethylene biguanide (PEB), polytetramethylene biguanide (PTMB), polyethylene hexamethylene biguanide (PEHMB), polymethylene biguanides (PMB), poly(allylbiguanidnio-co-allylamine), poly(N-vinylbiguanide), polyallybiguanide

The compounds can be synthesised in the laboratory by standard procedures or may be obtained from commercial suppliers, as will be well known to those skilled in the art.

PHMB, for example, may also have synonyms poly(hexamethylene)biguanide hydrochloride; polymeric biguanide hydrochloride; polyhexanide; biguanide; CAS Number 27083-27-8; 32289-58-0; IUPAC name Poly(iminoimidocarbonyl)iminohexamethylene hydrochloride. PHMB can be synthesised in the laboratory by standard procedures or may be obtained from suppliers, for example, Arch (http://www.archchemicals.com/Fed/BIO/Products/phmb.htm). Typically n=2 to 40, average n:11, average molecular weight: 3025. PHMB is sold as a biocide, for example for use in hygiene products, swimming pool water treatment and wound dressings.

Polyhexamethylene monoguanide (PHMG) can be synthesised in the laboratory by standard procedures or obtained from suppliers, for example from Shanghai Scunder Industry Co., Ltd, http://scunder.en.busytrade.com/products/info/683633/PHMG.html

As will be appreciated by those skilled in the art, the entry-promoting polymer may be a copolymer or heteropolymer, i.e. the monomers may not be intended to be identical. However, typically the monomer units may be intended to be identical.

The agent that inhibits antibacterial resistance and the entry-promoting agent may be covalently joined. Alternatively, the agent that inhibits antibacterial resistance and the entry-promoting agent may be provided as a formulation, for example as a non-covalent complex. The formulation may be prepared by mixing the entry-promoting agent and the agent that inhibits antibacterial resistance in appropriate ratios and under appropriate conditions of, for example, pH and salt concentration. The method may, for example, be performed from up to 100 fold molar excess of agent that inhibits antibacterial resistance over entry-promoting agent, through using an equal molar concentration of carrier and cargo molecules, to up to 1000 fold molar excess of entry-promoting agent over agent that inhibits antibacterial resistance. For example, an appropriate molar ratio of agent that inhibits antibacterial resistance and entry-promoting agent may be in the range of 1:0.1 to 1:50 or 1:0.5 to 1:1000, for example 1:1 to 1:10 or 1:5, for example around 1:1.5. An appropriate weight:weight ratio of agent that inhibits antibacterial resistance and entry-promoting agent may be in the range of 1:0.1 to 1:50 or 1:0.5 to 1:1000, for example 1:1 to 1:10 or 1:5, for example around 1:1.5. The formation of complexes is discussed further below.

The pH at which the entry-promoting agent and the agent that inhibits antibacterial resistance are mixed/incubated may be a high pH, for example 10-13.5, as discussed further below. It is particularly preferred that when the agent that inhibits antibacterial resistance is a nucleic acid, the agents are mixed/incubated at a high pH.

Alternatively, the pH at which the entry-promoting agent and the agent that inhibits antibacterial resistance are mixed/incubated may be a neutral pH, for example 6.5 to 8.6. It is particularly preferred that when the agent that inhibits antibacterial resistance is a peptide, the agents are mixed at a neutral pH. The appropriate incubation conditions will be identified by the skilled person and may be optimised according to the exact nature of the agents utilised. The conditions that provide most efficient delivery to the bacterial cell will be selected. In appropriate situations, the pH may be a low pH, i.e. less than 6.

In an embodiment, the entry promoting agent and the agent that inhibits antibacterial resistance may be provided together in a buffer having a high pH. Thus, the method for promoting entry of an agent that inhibits antibacterial resistance into a bacterial cell may comprise the step of exposing the bacterial cell to the agent that inhibits antibacterial resistance in the presence of an entry promoting agent (all as set out above) wherein the agent that inhibits antibacterial resistance and the entry promoting agent have been mixed or incubated at high pH, for example in a buffer having high pH. The term “high pH” will be well known to the skilled person and typically indicates a pH of above 9, for example above 9.5 or above 10, for example between 10 and 13.5. Typically the agent that inhibits antibacterial resistance and the entry promoting agent are mixed at high pH to form nanoparticles, before exposing the bacterial cell to the agent that inhibits antibacterial resistance in the presence of the entry promoting agent.

In a further embodiment, the entry promoting agent and the agent that inhibits antibacterial resistance may be provided together in a buffer having a neutral pH. Thus, the method for promoting entry of an agent that inhibits antibacterial resistance into a bacterial cell may comprise the step of exposing the bacterial cell to the agent that inhibits antibacterial resistance in the presence of an entry promoting agent (all as set out above) wherein the agent that inhibits antibacterial resistance and the entry promoting agent have been mixed or incubated at neutral pH, for example in a buffer having neutral pH. The term “neutral pH” will be well known to the skilled person and typically indicates a pH of around 7, for example above 6 or below 9, for example between 6.5 and 8.6. The agent that inhibits antibacterial resistance and the entry promoting agent may be mixed at neutral pH to form nanoparticles, before exposing the bacterial cell to the agent that inhibits antibacterial resistance in the presence of the entry promoting agent.

Specifically, buffers (with or without added salts, for example as commonly used in molecular biology buffers, for example PBS; NaCl; or many others) in the range of pH 6-13.5 are considered to provide formulations with improved delivery efficiencies, the pH is to be altered according to the nature of the agent to be delivered, as would be understood by a person of skill in the art. Resulting complex can be diluted 1:1 to 1:1000 in a suitable growth medium, even complex can be added at several time points to cells (repeated multiple transfection) to achieve more efficiency. The procedure involves separate dilution of both the entry promoting agent and the agent that inhibits antibacterial resistance in buffers with neutral to high pH and mixing them to form nanoparticles. Ratios and concentrations of the entry promoting agent and the agent that inhibits antibacterial resistance may be as discussed above in relation to preparation of a formulation and non-covalent complex; and in relation to the kit of parts.

Those skilled in the art will appreciate that high pH buffers can be easily prepared using, for example, NaOH or KOH. These buffer conditions provide improved transfection efficiencies when using typical complexation times, for example 30 minutes. Therefore, high pH buffers (and the entry promoting agents set out herein) can be easily incorporated into the protocols currently used by researchers.

Those skilled in the art will similarly appreciate that neutral pH buffers can be easily prepared by adjusting any appropriate buffer with HCl and/or NaOH or KOH. These buffer conditions provide improved transfection efficiencies when using typical complexation times, for example 30 minutes. Therefore, neutral pH buffers (and the entry promoting agents set out herein) can be easily incorporated into the protocols currently used by researchers.

A further aspect of the invention provides a method for preparing a complex comprising an entry promoting agent (for example PHMB or PHMG) and an agent that inhibits antibacterial resistance, the method comprising incubating the entry promoting agent and the agent that inhibits antibacterial resistance in a complexation buffer. The complexation buffer could for example be at a neural pH or at a high pH, depending on the agent. For example, the buffer could be at a neutral pH where the agent that inhibits antibacterial resistance is a peptide, or at a high pH when the agent is a nucleotide. Appropriate pH buffers are discussed above and optimal buffers would be determined by the skilled person for each agent. It is considered that nanoparticles are formed comprising the entry promoting agent (for example PHMB or PHMG) and the agent that inhibits antibacterial resistance, for example a peptide, nucleic acid or other polymer or a small molecule. The agent that inhibits antibacterial resistance could be an enzyme inhibitor. Specifically, formation of nanoparticles can be achieved by incubating PHMB and similar molecules as described above with the peptides, nucleic acids, or other polymer or small molecules in an appropriate buffer prior to use with cells. An appropriate incubation buffer may include water, PBS, and other buffers used commonly in laboratories. High pH buffers are described above. The optimal buffer may depend on the specific identity of both the entry promoting agent and the agent that inhibits antibacterial resistance, as will be apparent to those skilled in the art. Nanoparticle formation and bacterial cell delivery typically is achieved by dilution of both partner molecules in complexation buffer prior to mixing the two components. Also, mixing of the two components typically is carried out prior to combination with other excipients or active ingredients and application to bacterial cells including for use in vivo. Efficient nanoparticle formation is considered to occur within seconds or minutes but the procedure may be carried out over a number of hours. An appropriate ratio for efficient nanoparticle formation varies with different partner combinations. For example, 1-20:1 (wt:wt) for PHMB:plasmid DNA provides efficient nanoparticle formation. Further, 0.1:1 (wt:wt) up to 100:1 for PHMB:MecA peptide provides efficient nanoparticle formation (equal amount or excess of the carrier is preferred and works well). A person skilled in the art will be able to assess nanoparticle formation and delivery efficiencies when using different partner molecule ratios. Nanoparticle formation can be assessed in a number of ways. For example, an individual skilled in the art will be able to assess nanoparticle formation using dynamic light scattering (DLS) and microscopy methods.

A further aspect of the invention provides a complex comprising an entry promoting agent (for example PHMB or PHMG) and an agent that inhibits antibacterial resistance as defined herein, wherein the complex is obtainable (or obtained) by a method comprising incubating the entry promoting agent and the introduced agent in a complexation buffer, for example at a high or neutral pH, for example at a pH of 10-13.5 or at a pH of 6.5 to 8.6.

It is preferred if the entry-promoting agent is comprised in a nanoparticle. A nanoparticle has dimensions in the sub-micrometre range, such as in the nanometre range.

Agent that Inhibits Antibacterial Resistance

The agent that inhibits antibacterial resistance may be any agent that sensitizes an antibacterial-resistant bacterium to the effects of an antibacterial. For example, the inhibitory agent may bind to and inactivate a molecule within or produced by the bacterium that is responsible for resistance to an antibacterial or a class of antibacterials. The target molecule in the bacterium may, for example, be present inside the bacterial cell, in the periplasmic space or inside the bacterial cell wall. Preferably the agent that inhibits antibacterial resistance is a peptide, a nucleic acid, another polymer or a small molecule. The agent may be an enzyme inhibitor. For the avoidance of doubt the enzyme inhibitor may be a peptide or a nucleic acid. Typically, the enzyme inhibitor is a molecule, such as one with a molecular weight less than 40,000 Daltons.

The agent that inhibits antibacterial resistance may be a peptide aptamer or an RNA aptamer. The peptide aptamer may have a scaffold structure which enhances its structural stability, resistance to changes in pH, resistance to protease cleavage and temperature changes such as DARPin scaffolds based on ankyrin repeat proteins (Curr Opin Drug Discov Devel. 2007 March; 10(2):153-9., J Mol Biol. 2003 Sep. 12; 332(2):489-503.), Affimer based on a human Stefin A triple mutant (Woodman, R., Yeh, J. T-H., Laurenson, S. and Ko Ferrigno, P. Design and validation of a neutral scaffold for the presentation of peptide aptamers. J Mol Biol 352: 1118-1133 (2005).), or bicycle peptides based on chemically constrained peptides (Nature Chemical Biology 5, 502-507 (2009)).

The peptide agent that inhibits antibacterial resistance may be an antibody, antibody fragment or derivative either thereof. Thus, the antibody, antibody fragment or derivative thereof may specifically bind to and inhibit or sequester a target molecule (such as a protein) present in or associated with the bacterial cell or cell wall that is responsible for, or involved in the mechanism of, the antibacterial resistance, thus sensitising the bacterial cell to the antibacterial. By “antibody” we include substantially intact antibody molecules, as well as chimeric antibodies, humanised antibodies, human antibodies (wherein at least one amino acid is mutated relative to the naturally occurring human antibodies), single chain antibodies, bi-specific antibodies, antibody heavy chains, antibody light chains, homodimers and heterodimers of antibody heavy and/or light chains, and antigen binding fragments and derivatives of the same. We also include variants, fusions and derivatives of the antibodies and antigen-binding fragments thereof within the meaning of the terms “antibody” and “antigen-binding fragments thereof”. The term “antibody” also includes all classes of antibodies, including IgG, IgA, IgM, IgD and IgE. Thus, the antibody may be an IgG molecule, such as an IgG1, IgG2, IgG3, or IgG4 molecule. Preferably, the antibody of the invention is an IgG molecule, or an antigen-binding fragment, or variant, fusion or derivative thereof. More preferably the antibody is an IgG2 molecule. The antibody or fragment thereof may comprise or consist of an antigen-binding fragment selected from the group consisting of: an Fv fragment; an Fab fragment; and an Fab-like fragment. In a further embodiment, the Fv fragment may be a single chain Fv fragment or a disulphide-bonded Fv fragment. The Fab-like fragment may be an Fab′ fragment or an F(ab)₂ fragment. It is envisaged that the antibody or fragment thereof will be a monoclonal antibody or derived from a monoclonal antibody. Methods for generating monoclonal antibodies are well known in the art and include hybridoma production or the use of recombinant techniques.

The small molecule agent may be any appropriate organic molecule that inhibits a target molecule in a bacterial cell that is responsible or involved in a bacterial mechanism of antibacterial resistance. Thus, the agent knocks down the mechanism and sensitises the bacterial cell to the antibacterial in question.

Various different bacteria utilise a number of different mechanisms to resist killing by antibacterials. The different mechanisms involve different proteins or other targets in bacterial cells, depending on the bacterium and antibacterial in question. Examples of mechanisms of resistance to various classes of antibacterial drugs are provided in the following Tables 3 to 5, which also give examples of the bacteria that are resistant to these various classes of drugs. The agent that inhibits antibacterial resistance may be selected by reference to the antibacterial resistance determinants in these tables. Bockstael & Van Aerschot (2009) Eur. J. Med. 4(2), 141-155 also describes antibacterial resistance determinants in Table 2 on pp 146 and 147, the entire paper and Table 2 in particular is incorporated herein by reference.

For example, the inhibitory agent may be a peptide which binds to and inhibits the antibacterial resistance effect of the mecA gene product, Penicillin Binding Protein 2A (PBP2A), an altered penicillin binding protein which confers resistance to beta lactams. Peptides that bind to and inhibit antibacterial resistance determinants such as Penicillin Binding Proteins may be identified by methodologies which select peptides by virtue of their ability to bind to a target. Such methodologies include the use of phage display systems (see Willats (2002) Plant Mol. Biol. (2002) 50, 837-854 and Molek et al (2011) Molecules 16, 857-887, both incorporated herein by reference) (see also Example 1). Commercial phage display libraries and systems are available, for example from New England Biolabs. Example 1 provides a protocol for the preparation of PBP2a protein and the subsequent identification of binding moieties that bind to and inhibit PBP2a. Such binding proteins produced by these exemplified methods may be used in the methods of the invention.

A further system that may be used to identify suitable peptides is the CIS display system described in Odegrip et al (2004) Proc. Natl. Acad. Sci. USA (2004) 101, 2806-2810, incorporated herein by reference.

It is envisaged that the peptide agent that inhibits antibacterial resistance may be linear. Typically, peptides have from 50 to 400 amino acids. The peptides typically are in the molecular weight range 5.5 kDa to 40 kDa. It is particularly preferred if the peptide is less than 35 kDa in size.

By way of further example, the inhibitory agent may be a nucleic acid, such as single stranded DNA or RNA, which binds to and inhibits the metallo beta-lactamase from Klebsiella pneumoniae NDM-1 (also known as bla_{NDM-1}) (see Schlesinger et al (2011) Pharmaceuticals 4, 419-428, incorporated herein by reference). It is envisaged that the same targets suitable for targeting with peptides and peptide aptamers will also be suitable for targeting with RNA or modified RNA aptamers. Nucleic acids such as single stranded DNAs and RNAs that bind to and inhibit an antibacterial resistance determinant such as may be identified by using SELEX (systematic evolution of ligands by exponential enrichment) technology as described in Schlesinger et al (2011) Pharmaceuticals 4, 419-428, incorporated herein by reference; the SELEX approach is described in more detail in Tuerk & Gold (1990) Science 249, 505-510, incorporated herein by reference. See also Ellington & Szostak (1990) Nature 346, 818-822 which describes in vitro selection of RNA molecules that bind specific ligands, incorporated herein by reference. Typically, the nucleic acids are single stranded and have from 100 to 5000 bases.

It is preferred that the peptide comprises one or a combination of Sequence ID Nos. 1 to 4 or any peptide sequence having similar homology thereof. It is preferred that the peptide has at least 80% homology, more preferred at least 90% homology and most preferred at least 95% homology with these sequence IDs.

TABLE 3
Examples of Classes of Antimicrobials, Mechanisms of Resistance and Pathogens that can become Resistant to Drugs.

| | GENERAL | | |
|---|---|---|---|
| | MECHANISMS | | EXAMPLES of Pathogens and |
| ANTIMICROBIAL CLASS | OF | MECHANISM OF RESISTANCE TO DRUG | drugs that are overcome by |
| and examples of drugs in class | RESISTANCE | CLASS | resistance |
| Beta-lactams | Enzymatic | Destruction of beta-lactam rings by beta- | staphylococi to penicillin |
| Examples: oxacillin, penicillin, | destruction | lactamase enzymes. With the beta-lactam | Enterobacteriaceae to penicllins, |
| ampicillin, mezlocillin, peperacillin, | | ring destroyed, the antibacterial will no | cephalosporins, and aztreonam |
| cefazolin, cefotaxime, ceftazidime, | | longer have the ability to bind to PBP | |
| aztreonam, imipenem | | (Penicillin-binding protein), and interfere | |
| | | with cell wall synthesis. | |
| | Altered target | Changes in penicillin binding | Resistance of staphylococci to |
| | | proteins. Mutational changes in original | methicillin and oxacillin |
| | | PBPs or acquisition of different PBPs will | |
| | | lead to inability of the antibacterial to bind to | |
| | | the PBP and inhibit cell wall synthesis | |
| | Decreased | Porin channel formation is decreased. Since | Resistance of Enterobacter |
| | uptake | this is where beta-lactams cross the outer | aerogenes, Klebsiella |
| | | membrane to reach the PBP of Gram- | pneumoniae andPseudomonas |
| | | negative bacteria, a change in the number | aeruginosa to imipenem |
| | | or character of these channels can reduce | |
| | | beta-lactam uptake. | |
| Rifampicin | Inactivation | Phosphorylation, ADP-ribosyl group | Resistance of Nocardia |
| Examples: rifampicin | | transfer, glycosylation, oxidation | otitidiscaviarum to rifampicin |
| Aminoglyosides | Enzymatic | Modifying enzymes alter various sites on the | Resistance of many Gram-positive |
| Examples: gentamicin, tobramycin, | modification | aminoglycoside molecule so that the ability | and Gram negative bacteria to |
| amikacin, netilmicin, | | of this drug to bind the ribosome and halt | aminoglycosides |
| streptomycin, kanamycin | | protein synthesis is greatly diminished or | |
| | | lost entirely. | |
| Quinolones | Decreased | Alterations in the outer | Resistance of Gram negative and |
| Examples: ciprofloxacin, | uptake | membrane diminishes uptake of drug and/or | staphylococci (efflux mechanism only) |
| levofloxacin, norfloxacin, | | activation of an “efflux” pump that removes | to various quinolones |
| lomefloxacin | | quinolones before intracellular concentration | |
| | | is sufficient for inhibiting DNA metabolism. | |
| Chloramphenicol | Inactivation | Acetylation of drug by chloramphenicol | Enterococcus faecium and |
| | | acetyl transferase (CAT) | Pseudomonas aeruginosa strains are |
| | | | resistant to chloramphenicol. |
| Macrolides: | Inactivation | Hydrolysis (ereA, ereB), phosphorylation | Gram-positive bacteria (e.g. |
| Examples: Azithromycin, Clarithromycin, | | (mphA, | Streptococcus pneumoniae) and |
| Dirithromycin, Erythromycin, | | mphB, mphC), glycosylation (mtg) | Haemophilus influenzae |
| Roxithromycin, Telithromycin | | | |
| Lincosamides | Inactivation | Nucleotidyl group transfer (linA, linA′, linB) | staphylococci streptococci, and |
| Examples: Clindamycin,, Lincomycin, | | | Bacteroides fragilis |
| Pirlimycin | | | |
| Tetracyclines: | Inactivation | Oxidation of drug by TetX eliminates drug | Escherichia coli |
| Examples: Tetracycline, Chlortetracycline, | | activity | |
| Oxytetracycline | | | |
| Demeclocycline, Semi-synthetic, | Decreased | Efflux pump activities reduces intracellular | Escherichia coli, Campylobacter jejuni |
| Doxycycline, Lymecycline, | uptake | drug levels | and Streptococcus spp |
| Meclocycline, Minocycline, | | | |
| Rolitetracycline, Chlortetracycline, | | | |
| Tigecycline | | | |

Any of these mechanisms may be targeted with the methods of the invention.

TABLE 4
Examples of Classes of Antimicrobials, and Genes and Enzymes that
Confer Resistance in Pathogens

| ANTIMICROBIAL | GENERAL MECHANISMS | ENZYME AND |
|---|---|---|
| CLASS | OF RESISTANCE | GENE OR PROTEIN FAMILY |
| Beta-lactams | Enzymatic destruction | Lactamase: |
| | | Examples: CMY, TEM, SHV, CTX- |
| | | M, PER, VEB, GES, KPC, SME, |
| | | OXA, IMP, VIM, IND, NDM-1 (also |
| | | known as bla_{NDM-1} |
| | Altered target | Altered PBP |
| | | mecA |
| | Decreased uptake | Porin Channel mutations |
| | | penB |
| | | MexAB-OprM |
| Aminoglyosides | Enzymatic modification | acylation (AAC), |
| | | phosphorylation (APH), |
| | | nucleotidylation (ANT) |
| Quinolones | Increased efflux | Efflux pumps |
| | | NorA, PmrA, EmeA |
| Chloramphenicol | Inactivation | chloramphenicol acetyl transferase |
| | | (CAT) |
| Macrolides | Inactivation | Hydrolysis: (ereA, ereB), |
| | | phosphorylation (mphA, mphB, |
| | | mphC), glycosylation (mtg) |
| Lincosamides | Inactivation | Nucleotidyl group transfer (linA, |
| | | linA′, linB) |
| Tetracyclines | Inactivation | Oxidation of drug by TetX |
| | | eliminates drug activity |
| | Decreased uptake | Efflux pump activities reduces |
| | | intracellular drug levels |

It is envisaged that the invention may involve targeting any of the enzymes or gene products listed above.

By way of still further example, the inhibitory agent may be an enzyme inhibitor, such as a beta lactamase inhibitor or an efflux pump inhibitor or an amino glycoside kinase inhibitor as set out in Table 5. Clavulanic acid or a salt thereof is a suitable enzyme inhibitor which binds irreversibly to the active site of certain beta-lactamases.

TABLE 5
Antimicrobial Resistance Mechanisms and Compounds that can
Inhibit Resistance to Drugs

| Resistance Enzyme | Inhibitor |
|---|---|
| Lactamase | Clavulanate, Lactivicin |
| Efflux pumps | Capsanthin and capsorubin, carotenoids |
| | isolated from paprika; the flavonoids, |
| | rotenone, chrysin, phloretin and |
| | sakuranetin, Biricodar, Timcodar |
| Aminoglycoside kinases | Staurosporine, genistein, quercetin, |
| | isoquinoloine sulfonamides |

It is envisaged that any of the inhibitors listed in Table 5 may be utilised in the methods, compositions, kits and uses of the invention.

It is particularly preferred if the peptide or nucleic acid binds to and inhibits an antibacterial-resistance determinant selected from the group consisting of PBP2a, bla_{NDM-1} or Vim2.

It is preferred if the entry-promoting agent of the invention is polyhexamethylene biguanide (PHMB) or polyhexamethyleneguanide (PHMG) or an analogue or derivative either thereof.

The bacterium in any aspect of the invention that is to be sensitized to an antibacterial, and which may subsequently be killed by the antibacterial, may be Gram-negative, Gram-positive or may be a mycobacterium. The bacterium may be from the family Enterobacteriaceae, or it may be a Staphylococcus or a Steptococcus or may be from the genera Enterobacter, Klebsiella, Nocardia, Mycobacterium, Enterococcus, Pseudomonas, Bacteroides, Escherichia, Campylobacter. The bacterium is typically one of Klebsiella pneumoniae, Pseudomonas aeruginosa, Nocardia otitidiscaviarum, Mycobacterium tuberculosis, Enterococcus faecium, Streptococcus pneumoniae, Haemophilus unfluenzae, Bacteroides fragilis, Escherichia coli, Campylobacter jejuni.

The bacterium in any aspect of the invention may be resistant to one or more antibacterials selected from the group consisting of azithromycin, clarithromycin, dirithromycin, erythromycin, troleandomycin, roxithromycin, spiramycin, aztreonam, imipenem/cilastatin, meropenem, ertapenem, doripenem, panipenem/betamipron, biapenem, PZ-601, cefixime, cefdinir, cefditoren, cefoperazone, cefotaxime, cefpodoxime, ceftazidime, ceftibuten, ceftizoxime, ceftriaxone, cefepime, demeclocycline, doxycycline, minocycline, oxytetracycline, tetracycline, bacitracin, colistin, polymyxin B, ciprofloxacin, enoxacin, gatifloxacin, levofloxacin, lomefloxacin, moxifloxacin, norfloxacin, ofloxacin, and trovafloxacin.

In an embodiment of any aspect of the invention the bacterium is a multiple drug resistant (MDR) strain selected from the group consisting of Pseudomonas aeruginosa, Klebsiella pneumoniae, Burkholderia cepacia, Providencia stuartii or Acinetobacter baumannii that is resistant to one or more antibacterials selected from the group consisting of ciprofloxacin, meropenem, ceftazidime, aztreonam, azithromycin, clarithromycin, dirithromycin, erythromycin, roxithromycin, spiramycin, oxytetracycline and imipenem/cilastatin. In a further embodiment the bacterium is Staphylococcus aureus which is resistant to oxacillin.

It is envisaged that the bacterium may be a clinical strain or a clinical isolate.

The methods of the invention are suited to the situation wherein the bacterium is present in a human or animal and the method is used to combat bacterial infection of the human or animal. Thus, the invention includes a method of treating a human or animal infected with an antibacterial resistant bacterium wherein the human or animal is administered an effective amount of an agent that inhibits antibacterial resistance in combination with an entry-promoting agent and an antibacterial.

Thus, the invention also includes an effective amount of an agent that inhibits antibacterial resistance in combination with an entry-promoting agent and an antibacterial for use in treating a human or animal infected with an antibacterial resistant bacterium.

The invention also includes the use of an effective amount of an agent that inhibits antibacterial resistance in combination with an entry-promoting agent and an antibacterial in the manufacture of a medicament for treating a human or animal infected with an antibacterial resistant bacterium.

The medicament may further comprise a pharmaceutically acceptable excipient, adjuvant, diluent or carrier, as explained further below.

It is envisaged that the agent that inhibits antibacterial resistance may be for administration or administered simultaneously or sequentially with the entry-promoting agent, and simultaneously or sequentially with the antibacterial, as appropriate and in a manner to be determined by the physician. The agent that inhibits antibacterial resistance, entry-promoting agent and antibacterial may be formulated together or separately, as appropriate.

Typically, the bacterial infection is of an internal or external body surface selected from the group consisting of a surface in the oral cavity, the reproductive tract, the urinary tract, the respiratory tract, the gastrointestinal tract, the peritoneum, the middle ear, the prostate, vascular intima, the eye, including the conjunctiva or corneal tissue, lung tissue, heart valves, skin, scalp, nails, the interior of wounds or the surface of adrenal, hepatic, renal, pancreatic, pituitary, thyroid, immune, ovarian, testicular, prostate, endometrial, ocular, mammary, adipose, epithelial, endothelial, neural, muscle, pulmonary, dermis, epidermis or osseous tissue; or in a body fluid selected from blood, plasma, serum, cerebrospinal fluid, GI tract contents, sputum, pulmonary secretions and semen; or in or on body tissue selected from adrenal, hepatic, renal, pancreatic, brain, heart, pituitary, thyroid, immune, ovarian, testicular, prostate, endometrial, ocular, mammary, adipose, epithelial, endothelial, neural, muscle, pulmonary, epidermis and osseous tissue.

The patient to be treated with the methods, compositions/formulations and uses of the invention is envisaged to be a patient who is infected, suspected to be infected, or at risk of infection with a single or multi-drug resistant bacterium.

It is envisaged that the human or animal patient to be treated with the methods, compositions/formulations and uses of the invention may have a pre-established infection, they may be an immuno-compromised patient, a patient undergoing intensive or critical care, a patient suffering from trauma, a patient with a burn, a patient with an acute and/or chronic wound, a neonatal patient, an elderly patient, a patient with cancer, a patient suffering from an auto-immune condition, a patient with reduced or abrogated epithelial or endothelial secretion and/or secretion clearance or a patient fitted with a medical device. Of course, the methods and compositions/formulations may be used to treat any patient infected with antibacterial-resistant bacteria.

It will be appreciated that it may be desirable to identify the type of bacterium causing the infection and that it may be desirable to identify the nature of the antibacterial resistance of the bacterium. Thus, the invention includes a method of combating antibacterial-resistant bacterial infection of a human or animal as discussed above the method further comprising (a) identifying the type of bacterium and the nature of at least one antibacterial resistance determinant of the bacterium, (b) selecting an agent that inhibits the determined antibacterial resistance, (b) selecting an antibacterial to which the bacterium is resistant by virtue of the identified antibacterial resistance determinant and (c) administering to the human or animal, in the presence of an entry-promoting agent, the agent that inhibits the identified antibacterial resistance and the antibacterial.

The identification of the nature of the bacterial pathogen may be determined by any suitable method, such as by bacterial culture techniques and phylogeny. It is preferred that molecular techniques are employed to characterise the bacterial pathogen, such as by the use of DNA microarrays. An example of the identification and characterization of bacterial pathogens causing bloodstream infections by DNA microarray is described in Cleven et al (2006) J. Clin. Microbiol. 44(7), 2389-2397, incorporated herein by reference. This paper also describes the determination of antibacterial resistance. The nature of the antibacterial resistance determinant of an antibacterial resistant bacterium may be determined phenotypically by susceptibility testing but it is preferred if molecular techniques are employed such as microarray based detection. Microarray-based detection of 90 antibacterial resistance genes of Gram-positive bacteria is described in Perreten et al (2005) J. Clin. Microbiol. 43, 2291-2302, incorporated herein by reference.

It will be appreciated that more than one type of bacterium may be identified and that more than one type of antibacterial resistance may be identified. It will also be appreciated that it may be desirable to sensitize and subsequently kill more than one type of bacterium. Thus, the methods of the invention include the possibility of identifying, sensitizing to an antibacterial and killing with an appropriate antibacterial more than one type of bacterium.

In the first and second aspects of the invention it is preferred if the exposing of the bacterium, in the presence of an entry-promoting agent, to the agent that inhibits antibacterial resistance and the exposing of the bacterium to the antibacterial are done simultaneously. Conveniently, for example, an entry-promoting agent, the agent that inhibits antibacterial resistance and the antibacterial are present in the same composition. Also conveniently, the entry-promoting agent, the agent that inhibits antibacterial resistance and the antibacterial are present in a nanoparticle to which the bacterium is exposed. Conveniently, the bacterially infected human or animal is administered the nanoparticle to combat the infection. The nanoparticle may be comprised in a diluent or carrier or be present with other pharmaceutical excipients.

The methods of the invention are not restricted to the bacterium being present in or on a human or animal body. Therefore, in one embodiment the bacterium is present outside of a human or animal body, such as in or on inanimate material or on an inanimate surface, and the methods may be used to sensitize and kill bacteria as part of a disinfectant regime, such as in a hospital ward. Thus, the agents of the invention may be provided in a suitable liquid (i.e. aqueous or other solvent), a gaseous or solid form for use as a disinfectant. Such preparations may be administered in any appropriate manner such as in a liquid, mist, aerosol, vapour, powder, or crystalline form or any other suitable form, as would be understood by a person skilled in the art. Thus, the invention includes such preparations.

In a further aspect, the invention provides a composition/formulation comprising an entry-promoting agent and an agent that inhibits antibacterial resistance. Suitable agents are those provided above in relation to the earlier aspects.

In a yet further aspect, the invention provides a composition/formulation comprising an entry-promoting agent and an agent that inhibits antibacterial resistance for use in combating bacterial infection in a human or animal. Again, suitable agents are those provided above in relation to the earlier aspects.

In an embodiment of the preceding aspect, the human or animal may be administered an antibacterial. Indeed, the composition/formulation may further comprise an antibacterial. The antibacterial may be any antibacterial listed above.

The compositions of the preceding aspects of the invention may yet further comprise a pharmaceutically acceptable excipient, adjuvant, diluent or carrier.

By “pharmaceutically acceptable” is included that the formulation is sterile and pyrogen free. Suitable pharmaceutical carriers are well known in the art of pharmacy. The carrier(s) must be “acceptable” in the sense of being compatible with the agents of the invention and not deleterious to the recipients thereof. Typically, the carriers will be water or saline which will be sterile and pyrogen free; however, other acceptable carriers may be used.

The pharmaceutical composition/formulation of the invention may be formulated for intravenous, intramuscular, subcutaneous, oral, rectal, vaginal, nasal, ocular, or topical delivery to a human or animal patient. The route of administration and formulation will be selected based on the location and severity of the bacterial infection to be combated, as would be understood by a person of skill in the art.

Preferably, the pharmaceutical compositions/formulations of the present invention are a unit dosage containing a daily dose or unit, daily sub-dose or an appropriate fraction thereof, of the active ingredient(s).

The pharmaceutical compositions/formulations of the invention will normally be administered orally or by any parenteral route, in the form of a pharmaceutical formulation comprising the active ingredient(s), optionally in the form of a non-toxic organic, or inorganic, acid, or base, addition salt, in a pharmaceutically acceptable dosage form. Depending upon the infection and patient to be treated, as well as the route of administration, the compositions may be administered at varying doses.

In human therapy, the pharmaceutical compositions/formulations of the invention can be administered alone but will generally be administered in admixture with a suitable pharmaceutical excipient diluent or carrier selected with regard to the intended route of administration and standard pharmaceutical practice.

For example, the pharmaceutical compositions/formulations of the invention can be administered orally, buccally or sublingually in the form of tablets, capsules, ovules, elixirs, solutions or suspensions, which may contain flavouring or colouring agents, for immediate-, delayed- or controlled-release applications. The pharmaceutical compositions/formulations of invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The pharmaceutical compositions/formulations of the invention can also be administered parenterally, for example, intravenously, intra-arterially, intraperitoneally, intrathecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

The pharmaceutical compositions/formulations of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a pharmaceutical compositions/formulations of the invention and a suitable powder base such as lactose or starch.

Aerosol or dry powder formulations are preferably arranged so that each metered dose or “puff” contains at least 1 mg of a pharmaceutical composition/formulation of the invention for delivery to the patient. It will be appreciated that he overall daily dose with an aerosol will vary from patient to patient, and may be administered in a single dose or, more usually, in divided doses throughout the day.

Alternatively, the pharmaceutical compositions/formulations of the invention can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route, particularly for treating diseases of the eye.

For ophthalmic use, the pharmaceutical compositions/formulations of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the pharmaceutical compositions/formulations of the invention can be formulated as a suitable ointment containing the active compounds suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredients in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, oral or topical administration of the pharmaceutical compositions/formulations of the invention is the preferred route, being the most convenient. In circumstances where the recipient suffers from a swallowing disorder or from impairment of drug absorption after oral administration, the drug may be administered parenterally, e.g. sublingually or buccally.

For veterinary use, a pharmaceutical compositions/formulations of the invention is administered as a suitably acceptable formulation in accordance with normal veterinary practice and the veterinary surgeon will determine the dosing regimen and route of administration which will be most appropriate for a particular animal.

It is preferred that the patient is a human but the patient may be any other mammal that may benefit from the treatment. For example, the patient may be a mouse, a rat, a hamster, a rabbit, a cat, a dog, a goat, a sheep, a monkey or an ape.

A “therapeutically effective amount”, or “effective amount”, or “therapeutically effective”, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce or prevent a clinically significant deficit in the activity, function and response of the host. Alternatively, a therapeutically effective amount is sufficient to cause an improvement in a clinically significant condition in a host, for example a mammal.

The invention further provides a kit of parts comprising a plurality of agents that inhibit antibacterial resistance. The kit of parts may further comprise means for identifying bacteria and/or means for identifying antibacterial resistance determinants in bacteria. Such means may be those explained above in relation to the earlier aspects.

The kit of parts may further comprise one or more antibacterials. Suitable antibacterials may be those listed above in relation to the earlier aspects.

The kit of parts of the invention may yet further comprise an entry-promoting agent, preferably in combination with agents that inhibit antibacterial resistance. Suitable agents are listed above in relation to the earlier aspects.

The invention will now be described in more detail by reference to the following figures and non-limiting example:

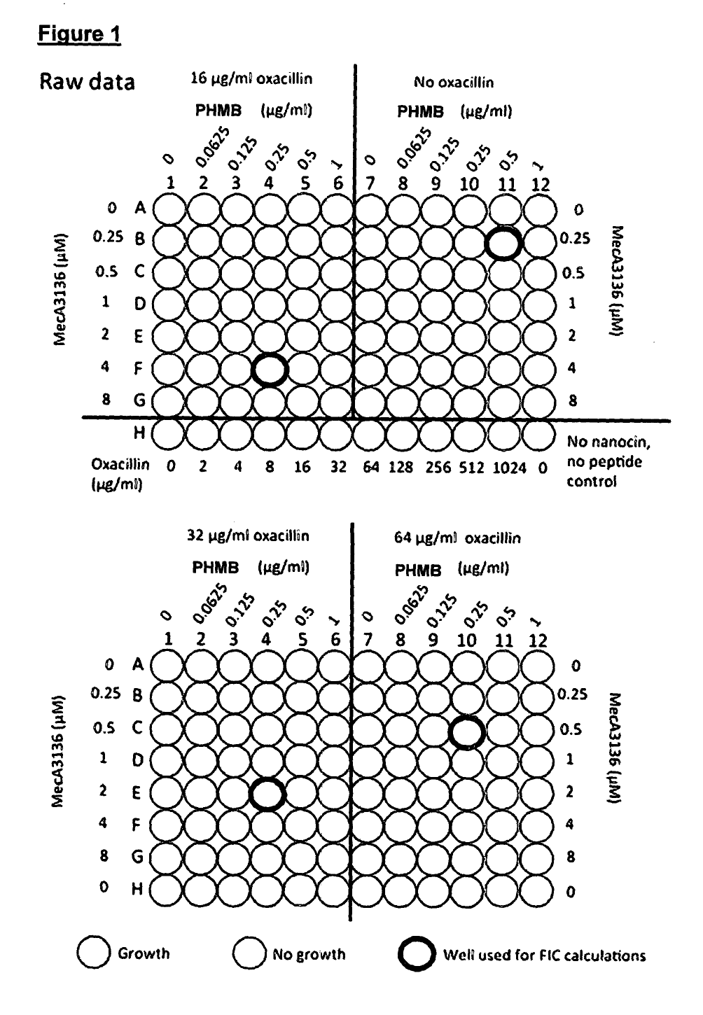

FIG. 1: FIC indices of PHMB and MecA peptides at different concentrations of oxacillin.

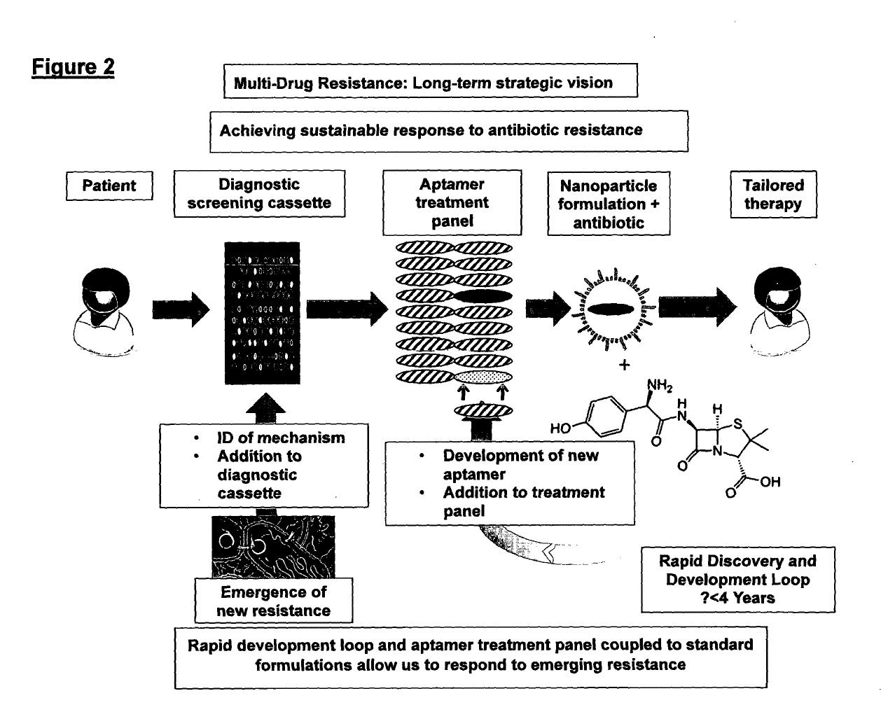

FIG. 2: Long-term strategic vision of implementation of invention.

### EXAMPLE 1

### Production of Recombinant PBP2a and Selection of Binding Agents Suitable for Inhibiting PBP2a

### PBP2a Production

Cloning of a Truncated mecA Gene.

Chromosomal DNA of MRSA (ATCC 3300) was used as a template for PCR. Primers were designed based on the published mecA sequence from the National Center for Biotechnology Information and the primers being a forward primer 5_-PBP2a-EcoRI, BamHI (5_-GGATCCGAATTC CTGGAAGTTCTGTTCCAGGGGCCCATGGCTTCAAAAGATAAA-3_) (Sequence ID No. 7) and a reverse primer 3_-PBP2a-XhoI,HindIII (5_-AAGCTTCTCGAGTTATTCATCTA TATCGTA-3_) (Sequence ID No. 8). The primers were designed so that the first 23 amino acids at the N terminus were deleted. The resulting DNA fragment L2 kb) was gel purified and then extracted by using a gel purification kit (Invitrogen) according to the manufacturer's protocol. The gene was ligated using T4 ligase into the pGEM-T vector (Promega, Madison, Wis.), and transformed into competent XL1-Blue cells. The mecA gene in the pGEM-T vector was sequenced by using T7 and SP6 promoter primers. The verified insert DNA and the pGEX-4T1 vector (GE Healthcare, Piscataway, N.J.) were both digested with the same restriction enzymes (EcoRI and XhoI) and then ligated together to give the expression vector pGEX-PBP2a.

PBP2a Expression.

The recombinant vector pGEX-PBP2a was transformed into Escherichia coli BL21(DE3) cells (Invitrogen) for protein expression. Cells were grown in Luria-Bertani broth containing 100_g/ml of ampicillin at 37° C., until the culture reached an optical density at 600 nm of 0.6. The culture was chilled in an ice bath for 10 min and then placed in a shaker at 18° C., and protein expression was induced by adding 0.5 mM IPTG (isopropyl-_-D-thiogalactopyranoside). Cells were then grown overnight (16 h) at 18° C. with shaking and then harvested by centrifugation at 4° C. For large-scale production, three 1-liter flasks each containing 350 ml of culture were used.

GST-PBP2a Purification.

All purification steps were performed at 4° C. The bacterial cell pellet was resuspended in 60 ml of cold lysis buffer (40 mM Na2HPO4, 10 mM KH2PO4, 300 mM NaCl [pH 7.4]). Bacterial cells were lysed using a Microfluidizer (model M100L; Microfluidics, Newton, Mass.). The bacterial extract was centrifuged for 30 min at 30,000_g, the supernatant was collected and spun for an additional 20 min, and the supernatant was again collected and stored frozen at _80° C. The GST-PBP2a fusion protein was purified on GST resin (GenScript catalog no. L00206) according to the manufacturer's instructions.

Cleavage of the GST Tag.

Thrombin (GE Healthcare, catalog no. 27-0846-01) was reconstituted with phosphate-buffered saline (PBS) to give a final solution of 1 U/_I. Small aliquots were stored at −80° C. To cleave the glutathione S-transferase (GST) tag from purified GST-PBP2a, 5 mg of GST-PBP2a was treated with 70 U of thrombin at 4° C. for overnight. SDS-PAGE was used to confirm cleavage. The free GST tag and uncleaved GST-PBP2a were removed from the cleaved product PBP2a by passage over the GST resin as described above. Purified untagged PBP2a passed though the column in the flowthrough, whereas GST and GST-PBP2a were retained. Fractions were collected, analyzed for purity by SDS-PAGE and for protein concentration by Bradford assay, and then concentrated.

Alternatively, PBP2a protein may be purchased from Sunny labs cat no. P555.

Characterization of Inhibition of PBP2a.

For inhibitor screening recombinantly expressed and purified PBP2a is immobilised using an affinity tag to the surface of the wells of a microtiter plate. The wells are then incubated with serially diluted solutions of the peptide aptamer or indeed any possible inhibitor of PBP2a, plus a PBP2a substrate, such as BIO-AMP (Bobba et al. 2011) at a fixed concentration in PBS. After approximately 15 min, the binding reactions are stopped, and the plates were developed as described (Bobba et al. 2011). The background levels of fluorescence from control reactions are subtracted and then the inhibitor binding data are calculated and plotted to determine the Ki^{app} of binding.

Reference—Sudheer Bobba, V. K. Chaithanya Ponnaluri, Mridul Mukherji and William G. Gutheil*Microtiter Plate-Based Assay for Inhibitors of Penicillin-Binding Protein 2a from Methicillin-Resistant Staphylococcus aureus. Antimicrob. Agents Chemother.June 2011 vol. 55 no. 6 2783-2787

Suitable Phage Display Protocol for Screening for Target Molecule (e.g. PBP2a) Binding Partners.

### Materials

EZ-Link® (Thermo Scientific Pierce, Cat. No. 21441)
DMSO (Dimethyl sulfoxide) (Sigma-Aldrich, Cat. No. D8418)
PBS (Phosphate Buffered Saline) (137 mM NaCl; 10 mM Phosphate; 2.7 mM KCl; pH 7.4)
PBST (PBS+0.1% Tween-20)
Spin Desalting Columns, 7K MWCO (Thermo Scientific Pierce, Cat. No. 89882/89883)
Nunc-Immuno™ MaxiSorp™ strips (Thermo Scientific, Cat. No. 469949)
10× Blocking Buffer (Sigma, Cat. No. B6429)
High Sensitivity Streptavidin-HRP (Thermo Scientific Pierce, Cat. No. 21130)
TMB (Seramun, Cat. No. S-001-TMB)
ER2738 E. coli cells
2TY media (per litre: 10 g yeast extract; 16 g tryptone; 5 g NaCl)
Tetracycline hydrochloride (12 mg/ml in 70% ethanol)
Streptavidin Coated (HBC) 8-well strips (Thermo Scientific Pierce, Cat. No. 15501)
0.2M Glycine, pH 2.2
1M Tris-HCl, pH 9.1
Triethylamine (Sigma-Aldrich, Cat. No. 10886)
1M Tris-HCl, pH 7
LB agar plates containing 100 μg/ml carbenicillin
Carbenicillin (500× stock: 50 mg/ml in ddH₂O)
M13K07 helper phage
Kanamycin (500× stock: 25 mg/ml in ddH₂O)
PEG-NaCl precipitation solution (20% (w/v) PEG 8000, 2.5M NaCl)
TE (10 mM Tris; 1 mM EDTA; pH 8.0)
Glycerol (Sigma-Aldrich, Cat. No. G6279)
Eppendorf Tubes (Eppendorf, Cat. No. 0030 108.116)
Streptavidin beads (Dynabeads® MyOne™ Streptavidin T1, 10 mg/ml) (Invitrogen Cat. No. 656.01/656.02)
Deep well 96 plate (Thermo Scientific, Cat. No. 95040450)
KingFisher (200 ul) 96 plates (Thermo Scientific, Cat. No. 97002540)
NeutrAvidin Coated (HBC) 8-well strips (Thermo Scientific Pierce, Cat. No. 15508)
DTT (Dithiothreitol)

### Methods

### First Panning Round

### Step 1: Crosslinking the Target Protein to Biotin

1. Equilibrate the vial of EZ-Link® NHS-SS-Biotin (Thermo Scientific Pierce, Cat. No. 21441) to room temperature before opening. Immediately before use, prepare a 5 mg/ml solution of NHS-SS-Biotin in DMSO (e.g. 0.5 mg of NHS-SS-Biotin in 100 μl of DMSO).
2. Add the appropriate volumes of NHS-SS-Biotin solution to protein—e.g. for a 12KDa protein add 5 μl of a 1 mg/ml solution to 0.4 μl of NHS-SS-Biotin in a total volume of 50 μl PBS (or PBST for hydrophobic proteins). Adjust the volume of protein to add depending on its molecular weight (MW)—i.e. add less protein with lower MW and more with higher MW proteins.
3. Incubate for 1 hour at room temp.
4. Desalt to remove any remaining biotin using Zeba Spin Desalting Columns, 7K MWCO (Thermo Scientific Pierce, Cat. No. 89882/89883) according to the manufacturer's instructions.
5. Aliquot and store at 4° C.

### Step 2: ELISA to Check Biotinylation

1. Aliquot 50 μl per well of PBS to Nunc-Immuno™ MaxiSorp™ strips (Thermo Scientific, Cat. No. 469949).
2. Add 1, 0.1 and 0.01 μl of biotinylated protein—i.e. add 1 μl of a 1:10 dilution for 0.1 μl and add 1 μl of a 1:100 dilution for 0.01 μl.
3. Incubate overnight at 4° C.
4. Wash 3× with 300 μl per well of PBST on a plate washer
5. Aliquot 250 μl per well of 10× Blocking Buffer (Sigma, Cat. No. B6429) and incubate at 37° C. for 3 hours.
6. Wash 3× with 300 μl per well of PBST on a plate washer
7. Dilute High Sensitivity Streptavidin-HRP (Thermo Scientific Pierce, Cat. No. 21130) 1:1000 in 2× Blocking Buffer (Sigma 10× Blocking Buffer diluted in PBST) and aliquot 50 μl per well.
8. Incubate for 1 hour at room temp on a vibrating platform shaker (Heidolph VIBRAMAX 100; speed setting 3).
9. Wash 6× with 300 μl per well of PBST on a plate washer
10. Aliquot 50 μl per well of TMB (SeramunBlau® fast TMB/substrate solution, Seramun, Cat. No. S-001-TME) and allow to develop. (Note the amount of time the plate is allowed to develop.)
11. Measure absorbance at 620 nm.
   Step 3: Setup Streptavidin Plates and ER2738 E. coli Cells, and Perform Phage Display
1. Pick a colony of ER2738 E. coli cells into 5 ml of 2TY media with 12 μg/ml tetracycline and incubate overnight in an orbital incubator at 37° C., 225 rpm.
2. Aliquot 300 μl per well of 2× Blocking Buffer into Streptavidin Coated (HBC) 8-well strips (Thermo Scientific Pierce, Cat. No. 15501) and incubate overnight (without agitation) at 37° C. setup 4 wells in total for each target protein (3 wells for pre-panning the phage and 1 well for binding the target protein and panning with phage).
3. Wash 3× with 300 μl per well of PBST on a plate washer
4. Aliquot 100 μl per well of 2× Blocking Buffer and add 2.5 μl of the biotinylated protein to the wells to be used for panning.
5. Incubate for 2 hours at room temp on the vibrating platform shaker (Heidolph VIBRAMAX 100; speed setting 3)—in the meantime, start pre-panning the phage.
6. Remove buffer from the first pre-pan well and add 100 μl of 2× Blocking Buffer. To this add 5 μl of preferred peptide display phage library expressing scaffold peptide of choice ie DARPIn, Affimer, linear. Mix and incubate on a vibrating platform shaker (Heidolph VIBRAMAX 100; speed setting 3) for 40 mins.
7. Remove buffer from the 2^{nd} pre-pan well and transfer the buffer containing the phage from the first pre-pan well to the 2^{nd} pre-pan well. Incubate for 40 mins, and then repeat for the 3^{rd} pre-pan well.
8. Wash 6× with 200 μl per well of PBST using a multichannel pipette the wells containing the target protein.
9. Transfer the phage from the pre-pan wells to the wells containing the target protein and incubate for 2 hours at room temp on a vibrating platform shaker (Heidolph VIBRAMAX 100; speed setting 3).
10. In the meantime, setup a fresh culture of ER2738 cells by diluting the overnight culture approx. 1:15 and incubating for approx. 1 hour at 37° C., 225 rpm to give an A₆₀₀ of □0.6.
11. Wash the panning well 6× in 300 μl per well of PBST on the plate washer
12. Elute the phage by adding 100 μl of 0.2M Glycine, pH 2.2, and incubating for 10 mins at room temp.
13. Neutralise by adding 15 μl of 1M Tris-HCl, pH 9.1. Mix and add immediately to an 8 ml aliquot of the ER2738 cells in a 50 ml falcon tube.
14. Dilute 14 μl of Triethylamine (Sigma-Aldrich, Cat. No. T0886) with 986 μl of PBS.
15. Elute any remaining phage by adding 100 μl of the diluted Triethylamine and incubating for 6 mins at room temp.
16. Neutralise by adding 50 μl of 1M Tris-HCl, pH7. Mix and add immediately to the ER2738 cells.
17. Incubate the cells for 1 hour at 37° C. (no shaking or shake at low speed, max. 90 rpm).
18. Plate 1 μl of the phage-infected E. coli K12 ER2738 cells onto LB agar plates containing 100 μg/ml

carbenicillin—incubate overnight at 37° C.
19. Centrifuge the remaining cells at 3,000×g for 5 mins to resuspend in a smaller volume and plate onto LB agar plates containing 100 μg/ml carbenicillin—incubate overnight at room temp.
20. Next day, count the colonies on the plates containing 1 μl of cells—multiply by 8,000 to determine the total number per 8 ml of cells (should be between 0.5−2×10⁶).
21. Scrape the cells from the remaining plates. To do this, add 5 ml of 2TY media+100 μg/ml carbenicillin to the plate, scrape using a disposable plastic spreader, transfer to a 50 ml falcon tube and mix. Add a further 2 ml of 2TY media+100 μg/ml carbenicillin to scrape any remaining cells.
22. Measure the absorbance at 600 nm of a 1:10 dilution to determine the dilution required for a 25 ml culture at A₆₀₀=0.2.
23. Dilute the cells in 2TY media+100 μg/ml carbenicillin in 125 ml glass flasks.
24. Incubate at 37° C., 230 rpm, for 1 hour.
25. Add 1 μl of M13K07 helper phage (titre ca. 10¹⁴/ml) and incubate at 37° C., 90 rpm, for 30 mins.
26. Add 50 μl of kanamycin (25 mg/ml) and incubate overnight in an orbital incubator at 25° C., 170 rpm.
27. Transfer the phage-infected cultures to 50 ml falcon tubes and centrifuge at 3,500×g for 10 mins.
28. Remove 500 μl of phage-containing supernatant for the second panning round. 29. Transfer the remaining supernatant to fresh tubes and add 6 ml of PEG-NaCl precipitation solution (20% (w/v) PEG 8000, 2.5M NaCl). Incubate for 2 hours at 4° C.
30. Centrifuge at 5,000×g for 20 mins to pellet the phage.
31. Pour off the supernatant (blotting the tube on tissue paper to remove all of the supernatant) and resuspend the pellet in 1 ml of TE.
32. Transfer to microcentrifuge tubes and centrifuge at 16,000×g for 10 mins. The supernatant contains the phage. Store at 4° C. or for long term storage, dilute with 40-50% glycerol and store at −80° C.

### Second Panning Round

1. Pick a colony of ER2738 E. coli cells into 5 ml of 2TY media with 12 μg/ml tetracycline and incubate overnight at 37° C., 225 rpm.
2. Wash 20 μl of Streptavidin beads (Dynabeads® MyOne™ Streptavidin T1, 10 mg/ml, Invitrogen Cat. No. 656.01/656.02) per target protein, 3 times in 500 μl of 2× Blocking Buffer using a magnet.
3. Resuspend in 100 μl of 2× Blocking Buffer per 20 μl of Streptavidin beads (or 200 μl minimum volume) and incubate overnight at room temp on a Stuart SB2 fixed speed rotator (20 rpm).
4. Centrifuge at 1,000×g for 1 min, immobilise the Streptavidin beads on a magnet and remove Blocking Buffer.
5. Replace with fresh 2× Blocking Buffer, resuspending in 100 μl per 20 μl of Streptavidin beads.
6. Pre-pan the phage: Mix 125 μl of phage-containing supernatant from the first panning round with 125 μl of 2× Blocking Buffer and add 50 μl of the pre-blocked Streptavidin beads—use Eppendorf Protein LoBind Tubes (Eppendorf, Cat. No. 0030 108.116). Incubate for 4 hours at room temp on the rotator.
7. At the same time, bind the target to the Streptavidin beads: Add 2.5 μl of the biotinylated target protein to 200 μl of 2× Blocking Buffer and 50 μl of the pre-blocked Streptavidin beads. Incubate for 4 hours at room temp on a Stuart SB2 rotator.
8. In the meantime, pre-block plates for the KingFisher Flex (Thermo Scientific):
   a. Pre-block enough wells in a deep well 96 plate (Thermo Scientific, Cat. No. 95040450) with 1 ml per well of 2× Blocking Buffer—this will be used for panning.
   b. Pre-block enough wells in two KingFisher (200 ul) 96 plates (Thermo Scientific, Cat. No. 97002540) with 300 μl per well of 2× Blocking Buffer—one will be used for eluting with Glycine, the other for eluting with Triethylamine.
      Block for 4 hours at 37° C.
9. Prepare sufficient wells in 4× deep well 96 plates with 950 μl per well of 2× Blocking Buffer—these will be used for the wash steps in the KingFisher protocol. Remove buffer from the pre-blocked elution plates. Aliquot 100 μl per well of 0.2M Glycine, pH 2.2, into one plate. Dilute 14 μl of Triethylamine with 986 μl of PBS and aliquot 100 μl per well into the other plate. Remove buffer from the pre-blocked deep well 96 plate.
10. Centrifuge the tubes containing the pre-panned phage and the biotinylated target at 1,000×g for 1 min and place on a magnet.
11. Wash the beads containing the biotinylated target protein 3 times in 500 μl of 2× Blocking Buffer.
12. Transfer the supernatant containing the phage to the beads containing the biotinylated target protein and resuspend. Transfer to the pre-blocked deep well 96 plate.
13. In the meantime, setup a fresh culture of ER2738 cells by diluting the overnight culture approx. 1:15 and incubating for approx. 1 hour at 37° C., 225 rpm.
14. The protocol will elute in Glycine for 10 mins. As soon as it has finished, neutralise by adding 15 μl of 1M Tris-HCl, pH 9.1. Mix and add to 8 ml aliquots of the ER2738 cells.
15. The protocol will then elute in Triethylamine for 6 mins. As soon as it has finished, neutralise by adding 50 μl of 1M Tris-HCl, pH7. Mix and add to the ER2738 cells.
16. Incubate the cells for 1 hour at 37° C. (no shaking or shake at low speed, max. 90 rpm). Plate and prepare phage as described for the first panning round, steps 17-31.

### THIRD Panning Round

1. Pick a colony of ER2738 E. coli cells into 5 ml of 2TY media with 12 μg/ml tetracycline and incubate overnight at 37° C., 225 rpm.
2. Aliquot 300 μl per well of 2× Blocking Buffer into NeutrAvidin Coated (HBC) 8-well strips (Thermo Scientific Pierce, Cat. No. 15508) and incubate overnight at 37° C. -setup 6 wells in total for each target protein (4 wells for pre-panning the phage, one for panning against the target protein, and a negative control for panning against a blank well).
3. Wash 3× with 300 μl per well of PBST on a plate washer
4. Aliquot 100 μl per well of 2× Blocking Buffer to the wells to be used for panning (one for panning against the target protein, and a negative control for panning against a blank well). Add 2.5 μl of the biotinylated protein to the well to be used for panning against the target protein. Incubate for 4 hours at room temp on a vibrating platform shaker (Heidolph VIBRAMAX 100; speed setting 3).
5. At the same time, start pre-panning the phage: Aliquot 20 μl of 10× Blocking Buffer into the first pre-panning well and add 200 μl of phage-containing supernatant from the 2^{nd} panning round. Incubate for 1 hour at room temp on a vibrating platform shaker (Heidolph VIBRAMAX 100; speed setting 3). Fill the remaining pre-panning wells with 200 μl per well of 2× Blocking Buffer.
6. Remove buffer from the 2^{nd} pre-pan well and transfer the contents of the first pre-panning well to the 2^{nd} pre-panning well. Incubate for another hour and repeat for the 3^{rd} and 4^{th} pre-panning wells.
7. In the meantime, setup a fresh culture of ER2738 cells by diluting the overnight culture approx. 1:15 and incubating for approx. 1 hour at 37° C., 225 rpm.
8. Wash 3× in PBST (manually) the wells containing the target protein and the negative control blank wells.
9. Transfer 100 μl per well of phage from the pre-pan wells to the wells containing the target protein and the negative control blank wells. Incubate for 30-45 mins at room temp on a vibrating platform shaker (Heidolph VIBRAMAX 100; max. speed setting 3).
10. Wash 6× with 300 μl per well of PBST on a plate washer
11. Elute the phage by adding 100 μl of 100 mM DTT and incubating for 20 mins at room temp.
12. Add to 8 ml aliquots of the ER2738 cells.
13. Incubate for 1 hour at 37° C. (no shaking or shake at low speed, max. 90 rpm). Plate and prepare phage as described for the first panning round, steps 17-31.

### EXAMPLE 2

### Sensitization of MRSA to Oxacillin Using MecA Inhibiting Peptides and PHMB

mecA encodes the protein Penicillin binding protein 2A (PBP2A) and is responsible for bacterial resistance to β-lactam antibacterials such as methicillin, penicillin, oxacillin, erythromycin, and tetracycline. Penicillin binding proteins (PBPs) are essential for bacterial cell wall synthesis. PBPs have been shown to catalyze a number of reactions involved in the process of synthesizing cross-linked peptidoglycan from lipid intermediates and mediating the removal of D-alanine from the precursor of peptidoglycan. PBPs bind β-lactam antibacterials because they are similar in chemical structure to the modular pieces that form the peptidoglycan. When they bind to penicillin, the β-lactam amide bond is ruptured to form a covalent bond with the catalytic serine residue at the PBPs active site. This is an irreversible reaction and inactivates the enzyme. Resistance to β-lactam antibacterials has come about through overproduction of PBPs and formation of PBPs that have low affinity for p-lactam antibacterials, as explained above. PBP2A has a low affinity for p-lactam antibacterials. Thus, expression of mecA reduces the effectiveness of 3-lactam antibacterials and enables the bacterium to make cells wall constituents unhindered.

The most commonly known carrier of mecA is methicillin-resistant Staphylococcus aureus (MRSA). It is also found in a number of other bacterial species, such as Streptococcus pneumoniae strains. In Staphylococcus species, mecA is spread on the SCCmec genetic element.

The following experiments exemplify the methods of the present invention. An MRSA strain is sensitised to oxacillin using an exemplary entry-promoting agent of the invention in combination with an agent that inhibits antibacterial resistance, and an antibacterial (oxacillin). The exemplary entry-promoting agent is PHMB. The exemplary agents that inhibit antibacterial resistance are MecA (i.e. PBP2A binding peptides) binding peptides MecA3136, MecA3140, HIW and E1. These peptides are Affimers based on the Stefin A scaffold (see Woodman, et al (2005) Design and validation of a neutral scaffold for the presentation of peptide aptamers. J Mol Biol 352: 1118-1133) and they have the following amino acid sequences:

MecA3136 (Sequence ID No. 1) has the
peptide aptamer sequence:
MHHHHHHSSGLVPCGWLKETAAAKFERQHMDSPDLSTWSHPQFEKGGGSG
GGSGGGSWSHPQFEKGTGSGGGSGGGSWSHPQFEKGTGSGGGSGGGSWSH
PQFEKGTGSGGGSGGGSWSHPQFEKGTDDDDKAMADIGSEMIPRGLSEAK
PATPEIQEIVDKVKPQLEEKTNETYGKLEAVQYKTQVLAKILTLGSTNYY
IKVRAGDNKYMHLKVFKSLALELSETPTPKAADRVLTGYQVDKNKDDELT
GF
(Sequence in bold denotes tag sequence)
MecA3140 (Sequence ID No. 2) has the
peptide aptamer sequence:
MHHHHHHSSGLVPCGWLKETAAAKFERQHMDSPDLSTWSHPQFEKGGGSG
GGSGGGSWSHPQFEKGTGSGGGSGGGSWSHPQFEKGTGSGGGSGGGSWSH
PQFEKGTGSGGGSGGGSWSHPQFEKGTDDDDKAMADIGSEMIPRGLSEAK
PATPEIQEIVDKVKPQLEEKTNETYGKLEAVQYKTQVLASTNYYIKVRAG
DNKYMHLKVFKSLFVVIQPERSNTWADRVLTGYQVDKNKDDELTGF
(Sequence in bold denotes tag sequence)
HIW (Sequence ID No. 3) has the
peptide aptamer sequence:
MHHHHHHSSGLVPCGWLKETAAAKFERQHMDSPDLSTWSHPQFEKGGGSG
GGSGGGSWSHPQFEKGTGSGGGSGGGSWSHPQFEKGTGSGGGSGGGSWSH
PQFEKGTGSGGGSGGGSWSHPQFEKGTDDDDKAMADIGSEMIPRGLSEAK
PATPEIQEIVDKVKPQLEEKTNETYGKLEAVQYKTQVLASTNYYIKVRAG
DNKYMHLKVFKSLHIWPITEIRRLVADRVLTGYQVDKNKDDELTGF
(Sequence in bold denotes tag sequence)
E1 (Sequence ID No. 4) has the
peptide aptamer sequence:
ASAATGVRAVPGNENSLEIEELARFAVDEHNKKENALLEFVRVVKAKEQH
WKAKLGHDTMYYLTLEAKDGGKKKLYEAKVWVKLIPTKDFHLNFKELQEF
KPVGDAAAAHHHHHH
Also disclosed are peptide sequences
Red1 and Red2 which were selected for
further experimental investigations and
they have the following amino acid
sequences:
Red1 (Sequence ID No. 5) has the
peptide aptamer sequence:
MHHHHHHSSGLVPCGWLKETAAAKFERQHMDSPDLSTWSHPQFEKGGGSG
GGSGGGSWSHPQFEKGTGSGGGSGGGSWSHPQFEKGTGSGGGSGGGSWSH
PQFEKGTGSGGGSGGGSWSHPQFEKGTDDDDKAMADIGSEMIPRGLSEAK
PATPEIQEIVDKVKPQLEEKTNETYGKLEAVQYKTQVLANLVGRISTNYY
IKVRAGDNKYMHLKVFNGPRQIKQVEWELIWADRVLTGYQVDKNKDDELT
GF*
(Sequence in bold denotes tag sequence)
Red2 (Sequence ID No. 6) has the
peptide aptamer sequence:
MHHHHHHSSGLVPCGWLKETAAAKFERQHMDSPDLSTWSHPQFEKGGGSG
GGSGGGSWSHPQFEKGTGSGGGSGGGSWSHPQFEKGTGSGGGSGGGSWSH
PQFEKGTGSGGGSGGGSWSHPQFEKGTDDDDKAMADIGSEMIPRGLSEAK
PATPEIQEIVDKVKPQLEEKTNETYGKLEAVQYKTQVLANLVGRISTNYY
IKVRAGDNKYMHLKVFKSLTPLEL*
(Sequence in bold denotes tag sequence)

MecA binding peptides MecA3136, MecA3140, HIW and E1 bind to MecA and block oxycillin binding to MecA in vitro.

The objectives of the experiments described in this example were to (1) determine minimum inhibitory concentrations (MICS) of PHMB and MecA peptides in an MRSA strain, and (2) to test for potentiation of oxacillin by using PHMB and the peptides.

HO 5096 0412 (EMRSA-15) is an MRSA strain endemic to UK hospitals. The EMRSA-15 genome consists of a single circular chromosome of 2,832,299 bp and a plasmid of 2473 bp. The fully annotated chromosome is available from the EMBL/GenBank databases with accession number HE681097. A preliminary gene prediction of the plasmid may be downloaded in Artemis format.

MRSA252 is a representative of the epidemic EMRSA-16 lineage endemic in UK hospitals. The strain has been typed as sequence type (ST)36 by MLST. The MRSA252 genome is composed of a single circular chromosome of 2,902,619 bp. The fully annotated genome is available from the EMBL/GenBank databases with accession number BX571856.

### Method

### Minimum Inhibitory Concentration (MIC) of Drugs and Peptides

Antibacterial susceptibility test of EMRSA-15 was carried out according to NCCLS guidelines for broth micro broth dilution with modifications. EMRSA-15 was streaked from a −70° C. glycerol stock to Columbia base agar (Oxoid) supplemented with 5% horse blood (Oxoid) and 6 μg/ml oxacillin (Sigma), and incubated for 18-20 h at 33° C. Two to four bacterial colonies were inoculated into 3 ml Mueller Hinton broth (MHB, Oxoid) supplemented with 6 μg/ml oxacillin (Sigma) and incubated for 18-20 h at 33° C. with shaking at 180 rpm. Cultures were adjusted to OD₆₂₅ 0.08-0.1 (equivalent to 0.5 Mcfarland or 1-2×10⁸ cfu/ml) with MHB and further diluted 1/50 to achieve a final concentration of 5×10⁵ cfu per well. Susceptibility to either oxacillin, PHMB or peptides was tested by adding oxacillin (0-1024 μg/ml in MHB), PHMB (0-512 μg/ml in 1×PBS) or peptide (0-512 μg/ml in 1×PBS) to 75 μl of culture in a final volume of 150 μl per well. Plates were incubated at 33° C. for 24 h and growth was scored visually.

### Synergy of PHMB and MecA Peptides

Synergy of PHMB and peptides (MecA3136, MecA3140) were tested by a standard checkerboard method of two-fold dilutions. PHMB and peptide were mixed at appropriate concentrations to a final volume of 30 μl in each well of a 96 well plate, and the mixtures were incubated at 20-22° C. for 30 mins before the addition of culture and oxacillin to a final volume of 150 μl. Plates were incubated and scored as above. FIC indices were calculated for wells without growth and containing the lowest combination of PHMB and peptide as follows: (A/MIC_{A})+(B/MIC_{B})=FIC_{A}+FIC_{B}=FIC index, where A is concentration of PHMB in combination, MIC_{A} is MIC of PHMB alone, B is concentration of peptide in combination, MIC_{B} is MIC of peptide alone. A control plate without oxacillin, and control wells of culture with increasing oxcaillin but without PHMB and peptide were included.

### Results

TABLE 6
MIC of EMRSA-15 to PHMB or MecA peptides (Strep-tagged)

| Growth media | PHMB | MecA3136 | MecA3140 |
|---|---|---|---|
| No oxacillin | 0.5 μg/ml | >512 μg/ml* | >512 μg/ml* |
| With oxacillin^{§} | 0.5 μg/ml | >176 μg/ml* | >176 μg/ml* |

^{§}Oxacillin used was 64 μg/ml
*Maximum concentration tested is shown

### Indications:

MIC of PHMB and MecA peptides did not change in the presence of oxacillin
PHMB will be tested at ½ MIC (e.g. 0.25 μg/ml) for delivery of MecA peptides (oxacillin sensitization assay)
MecA peptides do not inhibit growth of MRSA. It is possible that growth inhibition may occur at higher than tested concentrations, but this was not tested
Since MecA peptides do not have an MIC, arbitrary concentrations will be tested with PHMB (oxacillin sensitization assay)

TABLE 7
MIC of EMRSA-15 to oxacillin in the absence or
presence of PHMB or MecA peptide

| Growth Media | Oxacillin |
|---|---|
| MHB | 128 μg/ml |
| MHB + 0.25 μg/ml PHMB | 256 ug/ml |
| MHB + 176 μg/ml MecA3136 | 128 μg/ml |
| MHB + 176 μg/ml MecA3140 | 128 μg/ml |

### Indications:

PHMB alone did not potentiate oxacillin (no reduction in MIC)
Peptides alone did not potentiate oxacillin (no reduction in MIC)
PHMB increased MIC of MRSA-15 to oxacillin by 2-fold, which is not considered significant and is an unexpected result.
However, this may indicate at certain concentrations, PHMB is either trapping oxacillin in nanoparticles and making them unavailable to bacteria, or it is upregulating the expression of mecA and providing increased resistance to oxacillin.
If PHMB is forming nanoparticles with oxacillin and potentially delivering it to bacterial cells, then a three-dimensional assay involving peptide, PHMB and oxacillin may not give clear results

TABLE 8
FIC indices of PHMB and MecA peptides at different concentrations
of oxacillin (refer to FIG. 1 for an example of raw data)

| | PHMB + | PHMB + |
|---|---|---|
| Growth media | MecA3136 | MecA3140 |
| MHB | 1 | 1 |
| MHB + 16 μg/ml oxacillin ( 1/16 MIC) | 0.68 | 1 |
| MHB + 32 μg/ml oxacillin (⅛ MIC) | 0.59 | 1 |
| MHB + 64 μg/ml oxacillin (¼ MIC) | 0.52 | 0.9 |

MIC of oxacillin in this set of experiments was 256 μg/ml
Since MecA peptides do not inhibit growth, for calculation of FIC index, MIC of peptides was set to 22 μM (or 512 μg/ml), the maximum concentration tested previously which did not have a growth inhibitory effect

### Indications:

PHMB and MecA3136 at certain concentrations potentiated oxacillin from having an MIC of 256 μg/ml to 516 μg/ml (16-fold reduction of MIC, see FIG. 1 also)
FIC<0.5 indicates synergy. Although PHMB and MecA peptides did not show synergy, a decreasing FIC with increasing oxacillin concentrations indicates sensitization when PHMB and MecA3136 are used, and specificity of peptide for MecA
These results suggest MecA3136 may be the more promising of the two

TABLE 9
Dose response of MecA3136 and oxacillin at 0.25 μg/ml PHMB.

| | Oxacillin concentration (ug/ml) | | | | | |
|---|---|---|---|---|---|---|
| MecA3136 (uM) | 0 | 16 | 32 | 64 | 128 | 256 |
| 0 | 1 | 1 | 1 | 1 | 1 | 0 |
| 0.25 | 1 | 1 | 1 | 1 | nd | nd |
| 0.5 | 1 | 1 | 1 | 0 | nd | nd |
| 1 | 1 | 1 | 1 | 0 | nd | nd |
| 2 | 1 | 1 | 0 | 0 | nd | nd |
| 4 | 1 | 0 | 0 | 0 | nd | nd |
| 8 | 1 | 0 | 1 | 0 | nd | nd |

Table: Growth inhibition of EMRSA-15 by oxacllin enhanced by MecA3136+ 0.25 ug/ml Nanocin

This experiment focused on the 0.25 μg/ml PHMB data set as the most informative as at higher concentrations PHMB itself may have an effect on sensitivity and may be bactericidal on its own at 0.5 μg/ml, although at 1 μg/ml this is not clear.

In Table 9, 1 is growth and 0 is no growth (nd=not done). A clear enhancement of oxacillin activity by MecA3136 is seen. The table shows a clear dose response. The growth at 8 μM MecA3136 and 32 μg/m oxacillin may be anomalous, but outside of this then we can see how MecA3136 is restoring sensitivity to oxacillin. At this concentration of PHMB apparent synergistic effects of MecA3136 on sensitivity to oxacillin can be seen.

Clear enhancement of oxacillin activity by HIW and E1 was also seen when similar assays to MecA3136 and MecA3140 were conducted for these additional aptamers.

TABLE 10
Growth of EMRSA-15 in oxacillin, HIW and 1 ug/ml PHMB.
1 indicates growth, 0 indicates no growth.
Growth at Nanocin 1 ug/ml

| | |
|---|---|
| HIW | Oxacillin (ug/ml) |

| (ug/ml) | 0 | 16 | 32 | 64 |
|---|---|---|---|---|
| 0 | 1 | 1 | 1 | 0 |
| 5 | 1 | 1 | 1 | 0 |
| 10 | 1 | 0 | 0 | 0 |

Sequence HIW in Table 10 shows a good effect during the assay.

TABLE 11
Growth EMRSA-15 Oxacillin. E1 and 1 ug/ml PHMB.
1 indicates growth and 0 indicates no growth

| | Oxacillin (ug/ml) | | | | | |
|---|---|---|---|---|---|---|
| E1 (ug/ml) | 0 | 8 | 16 | 32 | 64 | 128 |
| 0 | 1 | 1 | 1 | 1 | 1 | 0 |
| 5 | 1 | 1 | 1 | 1 | 0 | 0 |
| 10 | 1 | 1 | 1 | 1 | 0 | 0 |
| 20 | 1 | 1 | 1 | 0 | 0 | 0 |

Sequence E1 is a different scaffold to MecA3136 and MecA3140, but in Table 11 shows a modest effect during the assay.

1. A composition comprising a bacterium entry-promoting agent and an agent that inhibits antibacterial resistance, wherein the entry-promoting agent comprises, or is comprised in, a nanoparticle.

2. A composition according to claim 1, wherein the composition further comprises an antibacterial.

3. A composition according to claim 1 wherein the entry-promoting agent comprises or consists of a linear and/or branched or cyclic polymonoguanide/polyguanidine, polybiguanide, analogue or derivative thereof according to the following Formula 1a or formula 1b: 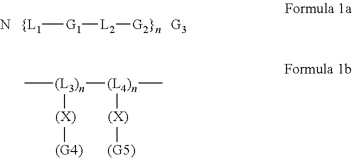
wherein:
“n”, refers to number of repeating units in the polymer, and n can vary from 2 to 1000, for example from 2 or 5 to 10, 20, 30, 40, 50, 60, 70, 80, 90, 100, 150, 200, 250, 300, 350, 400, 450, 500, 600, 700, 800 or 900;
G₁ and G₂ independently represent a cationic group comprising biguanide or guanidine, wherein L₁ and L₂ are directly joined to a Nitrogen atom of the guanide;
L₁ and L₂ are linking groups between the G₁ and G₂ cationic groups in the polymer and independently represent an aliphatic group containing C₁-C₁₄₀ carbon atoms, cycloaliphatic, heterocyclic, aromatic, aryl, alkylaryl, arylalkyl, or oxyalkylene radical; or a polyalkylene radical optionally interrupted by one or more, oxygen, nitrogen or sulphur atoms, functional groups or saturated or unsaturated cyclic moiety;
N and G₃ are optional end groups;
X can be either present or absent;
L₃, L₄ and X are linking groups between the G₄ and G₅ cationic groups in the polymer and independently represent an aliphatic group containing C₁-C₁₄₀ carbon atoms, cycloaliphatic, heterocyclic, aromatic, aryl, alkylaryl, arylalkyl, oxyalkylene radicals; or a polyalkylene radical optionally interrupted by one or more oxygen, nitrogen or sulphur atoms, functional groups as well as saturated or unsaturated cyclic moiety;
“G₄” and “G₅” are cationic moieties and can be same or different, and at least one of them is a biguanidine moiety or carbamoylguanidine, and the other moiety may be biguanidine or carbamoylguanidine or amine;
and cationic moieties G₄ and G₅ do not contain single guanidine groups.

4. A composition according to claim 3 wherein the entry-promoting agent according to Formula 1a comprises one or more of the following:

| Name | L₁ | G₁ | L₂ | G₂ |
|---|---|---|---|---|
| Polyhexamethylene biguanide | (CH₂)₆ | Biguanide | (CH₂)₆ | Biguanide |
| (PHMB) | | | | |
| Polyethylene biguanide (PEB) | (CH₂)₂ | Biguanide | (CH₂)₂ | Biguanide |
| Polyethylenetetramethylene | (CH₂)₂ | Biguanide | (CH₂)₄ | Biguanide |
| biguanide | | | | |
| Polyethylene hexamethylene | (CH₂)₂ | Biguanide | (CH₂)₆ | Biguanide |
| biguanide (PEHMB) | | | | |
| Polypropylene biguanide, | (CH₂)₃ | Biguanide | (CH₂)₃ | Biguanide |
| Polyaminopropyl | | | | |
| biguanide (PAPB) | | | | |
| Poly-[2-(2-ethoxy)-ethoxyethyl]- | (CH₂CH₂OCH₂CH₂OCH₂CH₂) | Biguanide | (CH₂CH₂OCH₂CH₂OCH₂CH₂) | Biguanide |
| biguanide-chloride] | | | | |
| (PEEG) | | | | |
| Polypropylenehexamethylene | (CH₂)₃ | Biguanide | (CH₂)₆ | Biguanide |
| biguanide | | | | |
| Polyethyleneoctamethylene | (CH₂)₂ | Biguanide | (CH₂)₈ | Biguanide |
| biguanide | | | | |
| Polyethylenedecamethylene | (CH₂)₂ | Biguanide | (CH₂)₁₀ | Biguanide |
| biguanide | | | | |
| Polyethylenedodecamethylene | (CH₂)₂ | Biguanide | (CH₂)₁₂ | Biguanide |
| biguanide | | | | |
| Polytetramethylenehexamethylene | (CH₂)₄ | Biguanide | (CH₂)₆ | Biguanide |
| biguanide | | | | |
| Polytetramethylenebiguanide | (CH₂)₄ | Biguanide | (CH₂)₄ | Biguanide |
| Polypropyleneoctamethylene | (CH₂)₃ | Biguanide | (CH₂)₈ | Biguanide |
| biguanide | | | | |
| Polytetramethyleneoctamethylene | (CH₂)₄ | Biguanide | (CH₂)₈ | Biguanide |
| Biguanide | | | | |
| Polyhexamethylene | (CH₂)₆ | Biguanide | CH₂—CH₂—NH—CH₂—CH₂ | Biguanide |
| diethylenetriamine biguanide | | | | |
| Polyhexamethylene guanide | (CH₂)₆ | guanidine | (CH₂)₆ | guanidine |
| (PHMG) | | | | |
| Polyethylene guanide | (CH₂)₂ | guanidine | (CH₂)₂ | guanidine |
| Polyethylenetetramethylene guanide | (CH₂)₂ | guanidine | (CH₂)₄ | guanidine |
| Polyethylene hexamethylene | (CH₂)₂ | guanidine | (CH₂)₆ | guanidine |
| guanide | | | | |
| Polypropylene guanide, | (CH₂)₃ | guanidine | (CH₂)₃ | guanidine |
| Polyaminopropyl guanide (PAPB) | | | | |
| Poly-[2-(2-ethoxy)-ethoxyethyl]- | (CH₂CH₂OCH₂CH₂OCH₂CH₂) | guanidine | (CH₂CH₂OCH₂CH₂OCH₂CH₂) | guanidine |
| guanide | | | | |
| Polypropylenehexamethylene | (CH₂)₃ | guanidine | (CH₂)₆ | guanidine |
| guanide | | | | |
| Polyethyleneoctamethylene guanide | (CH₂)₂ | guanidine | (CH₂)₈ | guanidine |
| Polyethylenedecamethylene guanide | (CH₂)₂ | guanidine | (CH₂)₁₀ | guanidine |
| Polyethylenedodecamethylene | (CH₂)₂ | guanidine | (CH₂)₁₂ | guanidine |
| guanide | | | | |
| Polytetramethylenehexamethylene | (CH₂)₄ | guanidine | (CH₂)₆ | guanidine |
| guanide | | | | |
| Polypropyleneoctamethylene | (CH₂)₃ | guanidine | (CH₂)₈ | guanidine |
| guanide | | | | |
| Polytetramethylene guanide | (CH₂)₄ | guanidine | (CH₂)₄ | guanidine |
| Polyhexamethylene | (CH₂)₆ | guanidine | CH₂—CH₂—NH—CH₂—CH₂ | guanidine |
| diethylenetriamine guanide | | | | |

or the entry promoting agent according to formula 1b comprises poly(allylbiguanidnio-co-allylamine), poly(N-vinylbiguanide), poly(allylcarbamoylguanidino-co-allylamine) or polyallybiguanide.

5. (canceled)

6. A composition according to claim 1, wherein the agent that inhibits antibacterial resistance is a peptide, a nucleic acid or a small molecule.

7. A composition according to claim 1, wherein the agent that inhibits antibacterial resistance is an enzyme inhibitor.

8. A composition according to claim 1, wherein the agent that inhibits antibacterial resistance is a peptide or nucleic acid which binds to and is capable of inhibiting an antibacterial-resistance determinant selected from the group consisting of PBP2a (gene product of mecA), NDM-1 or Vim2.

9. (canceled)

10. A composition according to claim 8, wherein the agent that inhibits antibacterial resistance is a peptide which comprises one or a combination of Sequence ID Nos. 1 to 4 or any peptide sequence having at least 90% homology thereof.

11. (canceled)

12. A composition according to claim 7 wherein the enzyme inhibitor is clavulanic acid or a salt thereof.

13. A composition according to claim 1 wherein the entry-promoting agent is polyhexamethylene biguanide (PHMB) or polyhexamethyleneguanide (PHMG) or analogues or derivatives thereof.

14. (canceled)

15. A composition according to claim 1 wherein the bacterium is Gram-negative, Gram-positive or is a mycobacterium.

16. A composition according to claim 1 wherein the bacterium is resistant to one or more antibacterials selected from the group consisting of azithromycin, clarithromycin, dirithromycin, erythromycin, troleandomycin, roxithromycin, spiramycin, aztreonam, imipenem, meropenem, ertapenem, doripenem, panipenem/betamipron, biapenem, PZ-601, cefixime, cefdinir, cefditoren, cefoperazone, cefotaxime, cefpodoxime, ceftazidime, ceftibuten, ceftizoxime, ceftriaxone, cefepime, demeclocycline, doxycycline, minocycline, oxytetracycline, tetracycline, bacitracin, colistin, polymyxin B, ciprofloxacin, enoxacin, gatifloxacin, levofloxacin, lomefloxacin, moxifloxacin, norfloxacin, ofloxacin, and trovafloxacin.

17. A composition according to claim 1 wherein the bacterium is a multiple drug resistant (MDR) strain selected from the group consisting of Pseudomonas aeruginosa, Klebsiella pneumoniae, Burkholderia cepacia, Staphylococcus aureus, Providencia stuartii and Acinetobacter baumannii, wherein said strain is resistant to one or more antibacterials selected from the group consisting of ciprofloxacin, meropenem, ceftazidime, aztreonam, azithromycin, methicillin, clarithromycin, dirithromycin, erythromycin, roxithromycin, spiramycin, oxytetracycline and imipenem/cilastatin.

18. A composition according to claim 1, for use in the treatment or prevention of a bacterial infection.

19. (canceled)

20. A method of combating antibacterial-resistant bacterial infection of a human or animal, the method comprising:
(a) identifying the type of bacterium and the nature of at least one antibacterial resistance determinant of the bacterium;
(b) selecting an agent that inhibits the determined antibacterial resistance;
(c) selecting an antibacterial to which the bacterium is resistant by virtue of the identified antibacterial resistance determinant; and
(d) administering to the human or animal, in the presence of an entry-promoting agent, the agent that inhibits the identified antibacterial resistance and the antibacterial.

21. A method according to claim 20, wherein the entry-promoting agent and agent that inhibits the identified antibacterial resistance are administered simultaneously, or sequentially with the antibacterial.

22. (canceled)

23. A method for sensitizing a bacterium to an antibacterial the method comprising the step of exposing the bacterium, in the presence of an entry-promoting agent, to an agent that inhibits antibacterial resistance.

24. A method according to claim 23, wherein the sensitizing of the bacterium to an antibacterial is for use in killing an antibacterial-resistant bacterium, the method further comprising the step of exposing the bacterium to an antibacterial.

25-27. (canceled)

28. A method according to claim 23 wherein the bacterium is present outside of a human or animal body, such as in or on inanimate material or on an inanimate surface.

29. A kit of parts comprising:
a) a plurality of agents that inhibit antibacterial resistance;
b) means for identifying bacteria and/or means for identifying antibacterial resistance determinants in bacteria;
c) one or more antibacterials; and
d) an entry-promoting agent, preferably in combination with agents that inhibit antibacterial resistance.

30-32. (canceled)
